(19) United States
(12) Patent Application Publication
(10) Pub. No.: US 2021/0278416 A1
(43) Pub. Date: Sep. 9, 2021

(54) GUT MICROBIOME FUNCTION PREDICTS RESPONSE TO ANTI-INTEGRIN BIOLOGIC THERAPY IN INFLAMMATORY BOWEL DISEASES

(71) Applicants: THE BROAD INSTITUTE, INC., CAMBRIDGE, MA (US); THE GENERAL HOSPITAL CORPORATION, BOSTON, MA (US)

(72) Inventors: ASHWIN N. ANANTHAKRISHNAN, BOSTON, MA (US); CHENGWEI LUO, CAMBRIDGE, MA (US); VIJAY YAJNIK, BOSTON, MA (US); RAMNIK J. XAVIER, BOSTON, MA (US)

(73) Assignees: THE BROAD INSTITUTE, INC., CAMBRIDGE, MA (US); THE GENERAL HOSPITAL CORPORATION, BOSTON, MA (US)

(21) Appl. No.: 16/612,141

(22) PCT Filed: May 9, 2018
(86) PCT No.: PCT/US2018/031793

§ 371 Date: Nov. 8, 2019

Related U.S. Application Data
(60) Provisional application No. 62/503,795, filed on May 9, 2017.

(51) Int. Cl. G01N 33/68

(52) U.S. Cl. CPC G01N 33/6893; G01N 2800/52; G01N 2800/065

(57) ABSTRACT

The present invention relates to a relationship between microbial metagenomic structure and function and clinical remission with anti-integrin therapy induction; longitudinal trajectory of changes in the microbiome with maintenance treatment; and a comprehensive predictive model incorporating clinical and microbiome-related data to accurately classify treatment response.

### INCORPORATION BY REFERENCE

This application claims priority to and benefit of U.S. Provisional Patent Application 62/503,795 filed May 9, 2017.

### FEDERAL FUNDING LEGEND

This invention was made with government support under Grant Nos, DK097142, DK43351 and DK92405 awarded by the National Institutes of Health. The government has certain rights in the invention.

The foregoing applications, and all documents cited therein or during their prosecution (“appln cited documents”) and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates generally to the gut microbiome and predictive responses to inflammatory bowel disease (IBD) pathogenesis and propagation and therapeutic methods involving the same.

### BACKGROUND OF THE INVENTION

Biologic monoclonal antibody therapy is the cornerstone of treatment of inflammatory diseases including inflammatory bowel diseases (IBD; Crohn's disease (CD), ulcerative colitis (UC)), rheumatoid arthritis (RA), and psoriasis (PsA) (Baumgart and Sandborn, 2012; Ordas et al., 2012; Ramiro et al., 2016; Singh et al., 2016) which affect over 10 million individuals in the United States (Brezinski et al., 2015; Cross et al., 2014; Molodecky et al., 2012). In each of these diseases, biologic therapy reduces disease-related morbidity (Baumgart and Sandborn, 2012; Ordas et al., 2012; Ramiro et al., 2016; Singh et al., 2016). Trials of therapeutic strategies have shown that early initiation of such therapy is associated with greater response (Castro-Rueda and Kavanaugh, 2008; D'Haens et al., 2008; Upchurch and Kay, 2012). Consequently, treatment paradigms have evolved from a step-up strategy to favor up-front biologic therapy to prevent damage. The availability of diverse therapeutic targets has brought forward the importance of personalizing treatment which require a priori predicting response to each mechanism of action. Initial attempts to do so relying on clinical factors yielded disappointing results (Siegel and Melmed, 2009). Genetics also performs imperfectly in predicting therapeutic response (Siegel and Melmed, 2009). Genomic expression profiles of target organs (intestine in IBD, articular cartilage in RA) demonstrated initial promise but predictive ability remains modest (Arijs et al, 2009), highlighting the need to identify novel determinants of response.

The past decade has highlighted the central role of the gut microbiome in many immune-mediated diseases (Becker et al., 2015; Forbes et 2016; Knights et al., 2013; Kostic et al., 2014). In IBD, the gut microbiome demonstrates reduced diversity, expansion of pro-inflammatory bacteria like Enterobacteriaceae and Fusobacteriaceae and depletion of phyla with anti-inflammatory effects such as Firmicutes (Becker et al., 2015; Knights et al., 2013; Kostic et al., 2014). Clinical observations of resolution of intestinal inflammation with fecal diversion and exacerbation following restoration of luminal continuity further support this concept (Rutgeerts et al., 1991). In RA, altered. T-lymphocyte response due to segmented filamentous bacteria (SFB) in the gut plays an important role (Wu et al., 2010). A similar reduction in diversity and depletion of Ruminococcus is seen in psoriatic arthritis as in IBD (Eppinga et al., 2014). Thus, given its role in the pathogenesis of these immune-mediated diseases, taxonomic and functional composition of the gut microbiome may influence likelihood of response to immuno-modulatory therapy for these diseases. An effect of the microbiome on therapy response has been demonstrated previously whereby inactivation of digoxin by Eggerthella lenta resulted in altered drug pharmacokinetics and reduced serum concentration (Haiser et al., 2013). Whether a similar effect may be seen with biologic therapy has not been defined previously.

Citation or identification of any document in this application is not an admission that such document is available as prior art to the present invention.

### SUMMARY OF THE INVENTION

The gut microbiome plays a central role in inflammatory bowel diseases (IBD) pathogenesis and propagation. To determine if the gut microbiome may predict responses to IBD therapy, Applicants conducted a prospective study with Crohn's disease (CD) or ulcerative colitis (UC) patients initiating anti-integrin therapy (vedolizumab). Disease activity and stool metagenomes at baseline, and weeks 14, 30, and 54 after therapy initiation were assessed. Community α-diversity was significantly higher, and Roseburia inulinivorans and a Burkholderiales species were more abundant at baseline among CD patients achieving week 14 remission. Several significant associations were identified with microbial function; 13 pathways including branched chain amino acid synthesis were significantly enriched in baseline samples from CD patients achieving remission. A neural network algorithm, vedoNet, incorporating microbiome and clinical data, and provided highest classifying power for clinical remission. Applicants hypothesize that the trajectory of early microbiome changes may be a marker of response to IBD treatment.

Using a prospectively recruited cohort of patients with IBD initiating gut-selective anti-integrin therapy with vedolizumab as a proof of concept, Applicants performed this study to (1) define the relationship between microbial metagenomic structure and function and clinical remission with vedolizumab induction; (2) to identify longitudinal trajectory of changes in the microbiome with maintenance treatment; and (3) develop a comprehensive predictive model incorporating clinical and microbiome-related data to accurately classify treatment response.

The present invention relates to a method for identifying and selecting a subject with an increased likelihood of responding to treatment of an inflammatory bowel disease (IBD), comprising measuring community α-diversity, Roseburia inulinivorans and/or a Burkholderiales species levels in a subject, wherein a subject with increased baseline levels of community α-diversity, Roseburia imiinivorans and/or a Burkholderiales species as compared to a subject with lower baseline levels of community α-diversity, Roseburia inulinivorans and/or a Burkholderiales species has an increased likelihood of responding to treatment for an IBD.

The IBD may be Crohn's disease (CD), ulcerative colitis (UC)), rheumatoid arthritis (RA), or psoriasis (PsA).

The present invention also includes measuring enrichment levels of one or more metabolic pathways, wherein the one or more metabolic pathways is A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of β-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; M, acetyl-CoA fermentation to butanoate II; N, colonic acid building blocks biosynthesis; O, lipid IVA biosysnthesis; P, N10-formyl-tetrahydrofolate biosysnthesis; and/or Q, pentose phosphate pathway; and/or R, pyruvate fermentation to acetate and lactate II; wherein a subject with enriched levels of A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of R-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; and/or M, acetyl-CoA fermentation to butanoate II as compared to a subject with lower baseline levels of A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of β-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; and/or M, acetyl-CoA fermentation to butanoate II has an likelihood of responding to treatment for CD; wherein a subject with enriched levels of N, colonic acid building blocks biosynthesis and/or O, lipid IVA biosysnthesis as compared to a subject with lower baseline levels of N, colonic acid building blocks biosynthesis and/or O, lipid IVA biosysnthesis has an increased likelihood of responding to treatment for UC and wherein a subject with depleted levels of N10-formyl-tetrahydrofolate biosysnthesis; and/or Q, pentose phosphate pathway; and/or R, pyruvate fermentation to acetate and lactate II as compared to a subject with higher baseline levels of N10-formyl-tetrahydrofolate biosysnthesis; and/or Q, pentose phosphate pathway; and/or R, pyruvate fermentation to acetate and lactate II has an increased likelihood of responding to treatment for UC.

The invention also includes treating the subject with an increased likelihood of responding to treatment of an inflammatory bowel disease (IBD) with an anti-integrin therapy. The anti-integrin therapy may be vedolizumab.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1A-1F: The differences of baseline stool samples between remission group and non-remission group. (a) The alpha-diversity measured in Fisher's alpha in remission and non-remission groups, segregated by diagnosis; (b) the beta-diversity measured by Bray-Curtis dissimilarity in intra- and inter-group fashion in remission and non-remission groups among CD and UC patients; (c-d) PCoA plots of baseline samples for CD and UC patients; and (e-f) the top 15 most abundant species in baseline samples for CD and UC patients. (box marks the interquartile range (IQR), the whiskers mark the range between lower quartile-1.5 IQR and higher quartile+1.5 IQR, and dots mark the outliers; *, q<0.1; **, q<0.01; ***, q<0.001; ns, not significant).

FIG. 2A-2B: The significantly differentiated taxa and pathways between remission and non-remission groups in baseline samples. (a) Two taxa, Burkholderiales and Roseburia inulinivorans, were significantly more abundant in CD remission baseline samples; and (b) pathways that were significantly differentiated between remission and non-remission groups in baseline samples for CD (left) and UC (right) patients (q<0.1). (Pathway codes: A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of β-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; M, acetyl-CoA fermentation to butanoate II; N, colonic acid building blocks biosynthesis; O, lipid IVA biosysnthesis; P, N10-formyl-tetrahydrofolate biosysnthesis; Q, pentose phosphate pathway; R, pyruvate fermentation to acetate and lactate II.).

FIG. 3A-3D: Longitudinal changes in taxa and pathways between remission and non-remission groups. (a-b) Log 2 fold change (log 2FC) in CD (a) and UC (b) patients' microbiome pathways that represented significant change at week 14 follow-up in comparison with baseline samples, divided into remission and non-remission groups (FDR<0.1); (c) log 2FC of species that represented significant change at week 14 follow-up in comparison with baseline sample (left panel, CD; right panel, UC), divided into remission and non-remission groups (FDR<0.1); and (d) the persistency index, P, for subjects with a later follow-up (wk30 or wk54) available. Horizontal bars indicate the t-test performed on respect group pair and the significance level (p<0.05: **, p<0.01; ***, p<0.001: ns, not significant). (Pathway codes: A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of R-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; M, acetyl-CoA fermentation to butanoate II; N, colonic acid building blocks biosynthesis; O, lipid IVA biosysnthesis; P, N10-formyl-tetrahydrofolate biosysnthesis; Q, pentose phosphate pathway; R, pyruvate fermentation to acetate and lactate II.).

FIG. 4: The architecture, training, and performance of vedoNet. (a) The vedoNet and associated other model variates (vedoNet.tx, vedoNet.hybrid, etc) are based on a neural network structure with an input layer, a few hidden layers with softmax dropout and rectified linear unit, and a binary output layer to classify if input data will support treatment outcome as remission or non-remission. The input data is a vector with two parts: the clinical metadata, and the microbiome profile which varies for different models (pathways, taxa, or a combination of both). The training deployed a 5-fold cross validation scheme, which resampled the subjects without replacement for test set and train set.

FIG. 5A. Related to FIG. 1: Differences in microbial diversity between remission group and non-remission group, at genus, family, class, and phylum levels.

FIG. 5B. Related to FIG. 1. Difference in the microbiome-dysbiosis index between remitters and non-remitters, by disease type

FIG. 6. Related to FIG. 2: Principle component analysis of baseline microbiome composition at class, family, and genus levels.

FIG. 7. Related to FIG. 4: Area under the curves of vedoNet.tx using profiles at different ranks as input.

FIG. 8. Related to FIG. 2: The unique SNPs that distinguished remission group and non-remission group among CD patients at baseline. Each row represent a SNP, contrasting remission (left panel) and non-remission (right panel) allele frequencies (color coded). The SNPs were grouped by species (vertical bars), and they were all from MetaCyc pathway PWY-5154 (L-arginine biosynthesis III).

FIG. 9: STAR Methods (Experimental model and subject details). Characteristics of the included patients initiating vedolizumab therapy

FIG. 10: Related to FIG. 3:The clinical variables used as vedoNet input

FIG. 11. Related to FIG. 3: The microbiome composition and pathway variables used as vedoNet input. Applicants selected the relative abundance of Roseburia imlinivorans, Burkholderiales, Eggerthella, Bifidobaterium longum, Ruminococcus gnavus, Veillonela parvula, Lactobacillus salivarius, and the relative abundance of pathways shown in the table based on the significant fold change difference between baseline and week 14 among the remission and non-remission groups according to the HUMANn2 pathway analysis.

FIG. 12: Related to FIG. 3: Performance of various models in classifying remitters and non-remitters to anti-TNF therapy in Crohn's disease and ulcerative colitis

### DETAILED DESCRIPTION OF THE INVENTION

The following detailed description is of example embodiments of the presently claimed invention with references to the accompanying drawings. Such description is intended to be illustrative and not limiting with respect to the scope of the present invention. Such embodiments are described in sufficient detail to enable one of ordinary skill in the art to practice the subject invention, and it will be understood that other embodiments may be practiced with some variations without departing from the spirit or scope of the subject invention.

A more diverse microbiome at baseline reflects prevalent microbes and/or metabolites with anti-inflammatory effect on colonic inflammation and a less disrupted mucosal barrier, leading to greater treatment response. Restoration of gut diversity with has been reported previously with anti-TNF therapy, though a more diverse microbiome has not been previously shown to be predictive of treatment response.

Taxonomically, the relative abundance of R. inulinivorans and Burkholderiales at baseline was predictive of week 14 remission. R. inulinivorans is a relatively low abundance gram-positive organism, certain strains of which encode genes for pro-inflammatory flagellin proteins that stimulate interleukin-8 production. R inulinivorans also produces butyrate and propionate both of which have anti-inflammatory effects through a variety of mechanisms including reinforcing the integrity of the colonic epithelial barrier, reducing oxidative stress, and decreasing inflammation through inhibition of nuclear factor κB (NF-κB) activation by histone deacetylation. Butyrate also inhibits inflammation through inhibition of the IFNγ/STAT1 signaling pathways associated with chronic inflammation and enhances apoptosis of colonic T-cells. Therefore, the present invention involves the determination of a relative abundance of R. inulinivorans and Burkholderiales at baseline. Measuring the relative abundance of R. inulinivorans and Burkholderiales is routine to one of skill in the art. In an advantageous embodiment, nucleic acids may be isolated from stool aliquots and shotgun sequencing may be utilized to characterize the relative abundance of R. inulinivorans and Burkholderiales.

In contrast to the relatively few changes between remitters and non-remitters at the species or genus level, differences in functional pathways were more striking. Pathways related to BCAA biosynthesis including citrulline, isoleucine, arginine and polyamine were enriched at baseline in CD patients who achieved week 14 remission. BCAA may reduce colonic inflammation through a variety of mechanisms. Arginine and isoleucine supplementation results in upregulation of human beta defensin 1 (hBD-1) in colon cells; reduced beta-defensin expression is associated with colonic inflammation in IBD. In a C57BL/6 mouse dextran sodium sulfate (DSS) colitis model, arginine supplementation reduced intestinal inflammation and cytokine production. Arginine is also a precursor for nitric oxide (NO) and endothelial NO is important for maintenance of intestinal perfusion and barrier integrity while NO produced by the inducible nitric-oxide synthase has direct anti-bacterial activity and is an important regulator of host defense. NO may also reduce damage from oxidative stress and through inhibition of NF-kB translocation. Glycosaminoglycan (GAG) degradation pathways were also enriched in those achieving remission. In mice models, intestinal flora mediated degradation of GAG resulted in metabolites with a cytotoxic effect on intestinal epithelium; inhibition of this degradation with antibiotics ameliorated colitis. Week 14 remission was associated with a reduction in several functional pathways up regulated at baseline. For example, the NAD salvage pathway decreased by week 14 among those achieving remission suggesting that clinical improvement was associated with a reduction in luminal oxidative stress. These discoveries together suggest that functional rather than taxonomic differences may be important determinants of treatment outcome.

Therefore, the present invention also involves detection of the pathway relative abundance which is routine to one of skill in the art. In an advantageous embodiment, nucleic acids may be isolated from stool aliquots and shotgun sequencing may be utilized to characterize pathway abundance. A persistent index was designed to measure the degree of persistency of the effect of treatment on taxa or pathways. This index was defined as the difference in the degree of the later follow-up sample mimicking the week 14 sample accounting for baseline differences. Formally, the index, P, was defined as:

P=[BCD(f,b)−BCD(f,k)]/BCD(k,b),

where BCD(x, y) represents the Bray-Curtis dissimilarity between sample x and y, and b, k, and f defined the baseline, week 14, and later follow-up (week 30 or week 54) samples. If sample f was identical to sample k representing maximum persistency, then P=1. If sample f was identical to sample b representing no persistency, then P=−1. A randomized profile was generated by decoupling the taxa and relative abundance and re-associating them at random. P was calculated for the randomized samples and independent student's t-test was applied to analyze differences between random and observed profiles.

Responders at week 14 demonstrated greater persistence of their microbial changes at 1 year compared to non-responders suggesting that early changes in the microbiome could be an indicator of clinical response. Similar clinical observations have been noted in CD and UC. In the ACT trial of infliximab in UC, endoscopic response by week 8 was associated with a lower rate of colectomy at week 54. In parallel, early reduction in fecal calprotectin has been associated with improved long-term outcomes in patients with IBD. Thus, early microbiome changes may be an added marker of sensitivity to treatment and initial response.

The present invention also encompasses a neural network algorithm (vedoNet) to predict treatment response. Use of available microbial taxa (vedoNet.tx; AUC 0.715) and pathways (vedoNet.pw, AUC 0.738) resulted in improved predictive ability. Taxonomic profiles at the level of genus, family, or class performed less well than information at the species level. A model incorporating clinical data, taxonomy, and pathway relative abundance without any pre-selection of variables performed better than each individual model (vedoNet.hybrid, AUC 0.776). Finally, a manually curated list of 40 microbiome variables provided the highest classifying power (AUC=0.872), successfully achieving >80% true positive discovery rate with a less than 25% false negative discovery rate. vedoNet was implemented in Python with Keras library; the codes, models, and tutorials can be found at www.bitbucket.org/luo-chengwei/vedoNet, the disclosures of which are incorporated by reference. Applicants repeated the analysis stratifying by type of IBD at baseline, allowing for incorporation of disease-specific phenotypic information. This did not significant improvement the predictive value of the vedoNet model for either CD (AUC 0.881) or UC (AUC 0.853). In analysis stratified by MD-index (above or below median), among high MD-index subjects, vedoNet's sensitivity was 0.75 and specificity was 0.769; while in low MD-index subjects, vedoNet's sensitivity and specificity were 0.818 and 0.85 respectively.

The invention also includes treating the subject with an increased likelihood of responding to treatment of an inflammatory bowel disease (IBD) with an anti-integrin therapy. The anti-integrin therapy may be vedolizumab.

The invention also includes methods of treating IBD with an anti-inflammatory drug, an immune system suppressor or an antibiotic. Anti-inflammatory drugs include, but are not limited to, aminosalicylates or corticosteroids. Immune system suppressors include, but are not limited to, azathioprine, mercaptopurine, cyclosporine, infliximab, adalimumab, golimumab, methotrexate, natalizumab, vedolizumab or ustekinumab. Antibiotics include, but are not limited to, metronidazole or ciprofloxacin. Other medications for treating IBD include, but are not limited to, anti-diarrheal medications, pain relievers, iron supplements, vitamin b-12 shots, calcium and vitamin D supplements or a special diet. Surgery may also be an option.

The present invention also includes targeting taxa and pathways that are significantly differentiated between remission and non-remission groups in baseline samples. For example, enhancement of community α-diversity, Roseburia inulinivorans and/or a Burkholderiales species may promote treatment of IBD. Targeted enhancement of community α-diversity, Roseburia inulinivorans and/or a Burkholderiales species via a small molecule or genetic engineering may be advantageous for the treatment of IBD. Similarly, targeted enhancement of varying metabolic pathways correlated to remission of IBD may also promote treatment of IBD. for example, targeted enhancement of particular genes in these pathways, such as by genetic engineering, may be advantageous for the treating IBD. Exemplary metabolic pathways include, but are not limited to, A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of β-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; M, acetyl-CoA fermentation to butanoate II: N, colonic acid building blocks biosynthesis; O, lipid IVA biosysnthesis; P, N10-formyl-tetrahydrofolate biosysnthesis; Q, pentose phosphate pathway; and R, pyruvate fermentation to acetate and lactate II.

Accordingly, the invention involves a non-human eukaryote, animal, mammal, primate, rodent, etc or cell thereof or tissue thereof that may be used as a disease model. As used herein, “disease” refers to a disease, disorder, or indication in a subject. For example, a method of the invention may be used to create a non-human eukaryote, e.g., an animal, mammal, primate, rodent or cell that comprises a modification, e.g., 3-50 modifications, in one or more nucleic acid sequences associated or correlated with a disease, e.g., an autoimmune disorder, such as inflammatory bowel disease (IBD), such as Crohn's disease or ulcerative colitisor cell or tissue of such. Such a mutated nucleic acid sequence be associated or correlated with an autoimmune disorder, such as inflammatory bowel disease (IBD), such as Crohn's disease or ulcerative colitis and may encode a disease associated protein sequence or may be a disease associated or correlated control sequence. The cell may be in vivo or ex vivo in the cases of multicellular organisms. In the instance where the cell is in cultured, a cell line may be established if appropriate culturing conditions are met and preferably if the cell is suitably adapted for this purpose (for instance a stem cell). Hence, cell lines are also envisaged. In some methods, the disease model can be used to study the effects of mutations on the animal or cell and development and/or progression of the disease using measures commonly used in the study of the disease. Alternatively, such a disease model is useful for studying the effect of a putatively pharmaceutically active compound or gene therapy on the disease. A disease-associated gene or polynucleotide can be modified to give rise to the disease in the model, and then putatively pharmaceutically active compound and/or gene therapy can be administered so as to observe whether disease development and/or progression is inhibited or reduced. In particular, the method comprises modifying so as to produce, one or more, advantageously 3-50 or more disease-associated or correlated gene(s) or polynucleotide(s). Accordingly, in some methods, a genetically modified animal may be compared with an animal predisposed to development of the disease, such that administering putative gene therapy, or pharmaceutically acceptable compound(s), or any combination thereof can be performed to assess how such putative therapy(ies) or treatment(s) may perform in a human.

Screening of such putative pharmaceutically active compound(s) and/or gene therapy(ies) can be by cellular function change and/or intracellular signaling or extracellular signaling change. Such screening can involve evaluating for dosages or dose curves, as well as combinations of potential drugs and/or therapies. An altered expression of one or more genome sequences associated with a signaling biochemical pathway can be determined by assaying for a difference in the mRNA levels of the corresponding genes between the disease model eukaryote or animal or cell or tissue thereof and a normal eukaryote, animal, tissue or cell, and to ascertain whether when the disease model is administered or contacted with a candidate chemical agent or gene therapy it reverts to or towards normal. An assay can be for mutation(s)-induced alteration in the level of mRNA transcripts or corresponding polynucleotides in comparison with such level(s) in a normal eukaryote or animal and whether such level(s) are placed towards or to normal when a therapy or treatment or agent is employed.

Inducing multiple mutations also enables the skilled person to divine new combinations of mutations that give rise to genetic disorders such as an autoimmune disorder, such as inflammatory bowel disease (IBD), such as Crohn's disease or ulcerative colitis. The ability to induce multiple mutations that accelerate or change the rate of a an autoimmune disorder, such as inflammatory bowel disease (IBD), such as Crohn's disease or ulcerative colitis accordingly provides many advantages heretofore unknown in research and development of pharmaceuticals, therapies and treatments for such disorders.

The present invention encompasses treating an inflammatory bowel disease (IBD), such as Crohn's disease or ulcerative colitis. Genome-wide association studies have generated insights into the mechanisms driving inflammatory bowel disease (IBD) and implicated genes shared by multiple autoimmune and autoinflammatory diseases (see, e.g., Graham and Xavier, 2013, Trends Immunol. 34, 371-378). GWAS studies have identified 163 loci associated with IBD (see, e.g., Jostins et al., 2012, Nature 491, 119-124, Khor et al. 2011, Nature 474, 307-317 and below table), any of which may be targeted in the present invention, alone or in combination.

| | | |
|---|---|---|
| Gene | Locus | Putative Function |

Select IBD Genes Identified by ImmunoChip [Jostins L, et al. Nature. 2012; 491: 119-124]

| | | |
|---|---|---|
| RNF186 | 1p36.13 | Highly expressed in intestine and contains a RING-type zinc finger that may |
| | | function as a ubiquitin ligase. Association with IBD has been validated in |
| | | several populations [Yang S K, et al. Inflammatory bowel diseases. 2013; |
| | | Juyal G, et al. PloS one. 2011; 6: e16565]. Evidence suggests genetic |
| | | interaction with another IBD gene, HNF4A [Garrison W D, et al. |
| | | Gastroenterology. 2006; 130: 1207-1220]. |
| SP110 | 2q37.1 | Associated with primary immunodeficiency. Expressed in hematopoietic cells |
| | | and contains a bromodomain with potential involvement in epigenetic |
| | | regulation. Loss of function mutations can decrease IL-10 production by B |
| | | cells [Bloch D B, et al. The Journal of allergy and clinical immunology. |
| | | 2012; 129: 1678-1680]. |
| SP140 | 2q37.1 | Expressed in hematopoietic cells and contains a bromodomain with potential |
| | | involvement in epigenetic regulation. |
| MST1 | 3p21 | Hepatocyte growth factor-like protein produced in the liver. Activates the |
| | | receptor tyrosine kinase MST1R on epithelial cells (and some subsets of |
| | | macrophages). Gain of function variants enhance macrophage motility |
| | | [Hauser F, et al. Genes and immunity. 2012; 13: 321-327]. |
| FUT2 | 19q13.3 | Golgi protein expressed in gastrointestinal tract. Enzymatic activity generates |
| | | a secreted oligosaccharide that functions as a substrate for synthesis of A and |
| | | B blood group antigens. Loss of function mutations (nonsecretor phenotype) |
| | | lack expression of blood group antigens in mucosal surfaces. Secretor status |
| | | correlates with alterations in the microbiome [Rausch P, et al. Proceedings of |
| | | the National Academy of Sciences of the United States of America. |
| | | 2011; 108: 19030-19035] and risk of IBD [Miyoshi J, et al. Journal of |
| | | gastroenterology. 2011; 46: 1056-1063] and T1D [Smyth D J, et al. Diabetes. |
| | | 2011; 60: 3081-3084]. |
| SLC22A4 | 5q31.1 | Ergothioneine transporter expressed in intestine and subsets of myeloid cells. |
| | | May regulate cellular redox state, potentially linking metabolism with |
| | | inflammatory responses [Kato Y, et al. Pharmaceutical research. |
| | | 2010; 27: 832-840]. |
| GSDMB | 17q12 | May be involved in regulation of epithelial cell apoptosis [Saeki N, et al. |
| | | Genes, chromosomes & cancer. 2009; 48: 261-271]. It is also highly expressed |
| | | in CD8 T cells. |
| ORMDL3 | 17q12 | Regulates ER stress response associated with inflammation [McGovern D P, |
| | | et al. Nature genetics. 2010; 42: 332-337]. |
| TNFSF15 | 9q32 | Expressed on endothelial cells and activated APCs. One of its receptors |
| | | (TNFRSF25) promotes Treg expansion in a ligand-dependent manner [Khan |
| | | S Q, et al. J Immunol. 2013; 190: 1540-1550]. |
| TNFAIP3 | 6q23 | Ubiquitin modifying enzyme expressed in myeloid cells. Negatively regulates |
| | | NFkB signaling and inflammatory cytokines [Hammer G E, et al. Nature |
| | | immunology. 2011; 12: 1184-1193]. |
| SLC6A7 | 5q32 | Proline transporter that may regulate cellular metabolic state and |
| | | inflammation. |
| IL10RA | 11q23 | Receptor for IL-10 broadly expressed on hematopoietic cells. Transduces |
| | | immunosuppressive signal through STAT3 and TYK2. Associated with early |
| | | onset IBD [Moran C J, et al. Inflammatory bowel diseases. 2013; 19: 115-123]. |

Select IBD Genes with Coding Variants Identified by Exome Sequencing [Rivas M A, et al.
Nature genetics. 2011; 43: 1066-1073]

| | | |
|---|---|---|
| IL23R | 1p31.3 | Receptor for IL-23 expressed predominantly in T cells. Promotes |
| | | differentiation of pathogenic Th17 cells [Ghoreschi K, et al. Nature. |
| | | 2010; 467: 967-971]. |
| CARD9 | 9q34.3 | Expressed in myeloid cells where it promotes activation of NFkB and |
| | | inflammatory cytokines downstream of pattern recognition receptors (PRRs) |
| | | that are associated with immunoreceptor tyrosine-based activation motifs |
| | | (ITAMs) or hemi-ITAMs [Hara H, et al. Nature immunology. 2007; 8: 619- |
| | | 629]. Promotes cytokine environment conducive to Th17 differentiation. |
| NOD2 | 16q21 | Intracellular PRR specific for bacterial peptidoglycans and is expressed in |
| | | myeloid cells. Activates NFkB and promotes inflammatory cytokines. Can |
| | | induce bacterial killing in an autophagy-dependent manner [Homer C R, et |
| | | al. Gastroenterology. 2010; 139: 1630-1641. 1641 e1631-1632]. |
| IL18RAP | 2q12 | Accessory protein for IL-18 receptor expressed on NK and T cells. Promotes |
| | | stimulatory effect of IL-18 on T cell IFN-γ production [Cheung H, et al. J |
| | | Immunol. 2005; 174: 5351-5357]. |
| MUC19 | 12q12 | Gel-forming mucin expressed in epithelial tissues. Potential role in barrier |
| | | function and interaction with microbial communities. |
| CUL2 | 10p11.21 | Component of E3 ubiquitin-protein ligase complex potentially linking |
| | | proteosomal system with autophagy. |
| PTPN22 | 1p13.2 | Protein tyrosine phosphatase that regulates T and B cell responses at the |
| | | level of antigen receptor signaling [Rhee I, Veillette A. Nature immunology. |
| | | 2012; 13: 439-447]. |
| Clorf106 | 1q32.1 | Expressed in epithelial cells of the gastrointestinal tract. May promote |
| | | epithelial integrity and barrier function. |

The present invention also involves targeting metabolic pathways, which are predictive of treatment of IBD. Metabolic pathways include, but are not limited to, A, super-pathway of arginine and polyamine biosynthesis; B, super-pathway of branched amino acid biosynthesis; C, Calvin-Benson-Bassham cycle; D, L-citrulline biosynthesis; E, dTDP-L-rhamnose biosynthesis I; F, super-pathway of N-acetyleglucosamine, N-acetylmannosamin and N-acetylneuraminate degradation; G, super-pathway of β-D-glucuronide and D-glucuronate degradation; H, super-pathway of hexitol degradation; I, L-isoleucine biosynthesis I; J, super-pathway of polyamine biosynthesis I; K, L-histidine degradation III; L, GDP-mannose biosynthesis; M, acetyl-CoA fermentation to butanoate II; N, colonic acid building blocks biosynthesis; O, lipid IVA biosysnthesis; P, N10-formyl-tetrahydrofolate biosysnthesis and/or Q, pentose phosphate pathway: R, pyruvate fermentation to acetate and lactate II.). Measurements of enrichment levels of these pathways are predictive as to the success of treatment of a patient diagnosed with IBD.

Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992): Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994). Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787). The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Packaging cells are typically used to form virus particles that are capable of infecting a host cell. Such cells include 293 cells, which package adenovirus, and % ψ2 cells or PA317 cells, which package retrovirus. Viral vectors used in gene therapy are usually generated by producer a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Accordingly, AAV is considered an ideal candidate for use as a transducing vector. Such AAV transducing vectors can comprise sufficient cis-acting functions to replicate in the presence of adenovirus or herpesvirus or poxvirus (e.g., vaccinia virus) helper functions provided in trans. Recombinant AAV (rAAV) can be used to carry exogenous genes into cells of a variety of lineages. In these vectors, the AAV cap and/or rep genes are deleted from the viral genome and replaced with a DNA segment of choice. Current AAV vectors may accommodate up to 4300 bases of inserted DNA. There are a number of ways to produce rAAV, and the invention provides rAAV and methods for preparing rAAV. For example, plasmid(s) containing or consisting essentially of the desired viral construct are transfected into AAV-infected cells. In addition, a second or additional helper plasmid is cotransfected into these cells to provide the AAV rep and/or cap genes which are obligatory for replication and packaging of the recombinant viral construct. Under these conditions, the rep and/or cap proteins of AAV act in trans to stimulate replication and packaging of the rAAV construct. Two to Three days after transfection, rAAV is harvested. Traditionally rAAV is harvested from the cells along with adenovirus. The contaminating adenovirus is then inactivated by heat treatment. In the instant invention, rAAV is advantageously harvested not from the cells themselves, but from cell supernatant. Accordingly, in an initial aspect the invention provides for preparing rAAV, and in addition to the foregoing, rAAV can be prepared by a method that comprises or consists essentially of: infecting susceptible cells with a rAAV containing exogenous DNA including DNA for expression, and helper virus (e.g., adenovirus, herpesvirus, poxvirus such as vaccinia virus) wherein the rAAV lacks functioning cap and/or rep (and the helper virus (e.g., adenovirus, herpesvirus, poxvirus such as vaccinia virus) provides the cap and/or rev function that the rAAV lacks); or infecting susceptible cells with a rAAV containing exogenous DNA including DNA for expression, wherein the recombinant lacks functioning cap and/or rep, and transfecting said cells with a plasmid supplying cap and/or rep function that the rAAV lacks; or infecting susceptible cells with a rAAV containing exogenous DNA including DNA for expression, wherein the recombinant lacks functioning cap and/or rep, wherein said cells supply cap and/or rep function that the recombinant lacks; or transfecting the susceptible cells with an AAV lacking functioning cap and/or rep and plasmids for inserting exogenous DNA into the recombinant so that the exogenous DNA is expressed by the recombinant and for supplying rep and/or cap functions whereby transfection results in an rAAV containing the exogenous DNA including DNA for expression that lacks functioning cap and/or rep. The rAAV can be from an AAV as herein described, and advantageously can be an rAAV1, rAAV2, AAV5 or rAAV having hybrid or capsid which may comprise AAV1, AAV2, AAV5 or any combination thereof. One can select the AAV of the rAAV with regard to the cells to be targeted by the rAAV. In addition to 293 cells, other cells that can be used in the practice of the invention and the relative infectivity of certain AAV serotypes in vitro as to these cells; see Grimm, D. et al, J. Virol. 82: 5887-5911 (2008) Aerosolized delivery is preferred for AAV or adenovirus delivery in general. An adenovirus or an AAV particle may be used for delivery. Suitable gene constructs, each operably linked to one or more regulatory sequences, may be cloned into the delivery vector. RNA(s) can be delivered using particles, adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For examples, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses may be based on or extrapolated to an average 70 kg individual, and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The vectors can be injected into the tissue of interest.

Among vectors that may be used in the practice of the invention, integration in the host genome is possible with the retrovirus, lentivirus, and adeno-associated virus gene transfer methods, often resulting in long term expression of the inserted transgene. Additionally, high transduction efficiencies have been observed in many different cell types and target tissues. The tropism of a retrovirus can be altered by incorporating foreign envelope proteins, expanding the potential target population of target cells. Lentiviral vectors are retroviral vectors (and hence both lentiviral and retroviral vectors may be used in the practice of the invention). Moreover, lentiviral vectors are preferred as they are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors that may be used in the practice of the invention include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990): Wilson et al., J. Virol. 63:2374-2378 (1989); Miller et al., J. Virol. 65:2220-2224 (1991); PCT/US94/05700). Zou et al. administered about 10 μl of a recombinant lentivirus having a titer of 1×10⁹ transducing units (TU)/ml by an intrathecal catheter. These sort of dosages can be adapted or extrapolated to use of a retroviral or lentiviral vector in the present invention.

Also useful in the practice of the invention is a minimal non-primate lentiviral vector, such as a lentiviral vector based on the equine infectious anemia virus (EIAV) (see, e.g., Balagaan, J Gene Med 2006; 8: 275-285, Published online 21 Nov. 2005 in Wiley InterScience (www.interscience.wiley.com). DOI: 10.1002/jgm.845). The vectors may have cytomegalovirus (CMV) promoter driving expression of the target gene. Intracameral, subretinal, intraocular and intravitreal injections are all within the ambit of the instant invention (see, e.g., Balagaan, J Gene Med 2006; 8: 275-285, Published online 21 Nov. 2005 in Wiley InterScience (www.interscience.wiley.com). DOI: 10.1002/jgm.845). In this regard, mention is made of RetinoStat@, an equine infectious anemia virus-based lentiviral gene therapy vector that expresses angiostatic proteins endostain and angiostatin that is delivered via a subretinal injection for the treatment of the web form of age-related macular degeneration is also contemplated (see, e.g., Binley et al., HUMAN GENE THERAPY 23:980-991 (September 2012)). Such a vector may be modified for practice of the present invention. Dosing of RetinoStat® (e.g., 1.1×10⁵ transducing units per eye (TU/eye) in a total volume of 100 sW) can be applied or extrapolated from in practicing the present invention with a lentivirus.

The invention also can be practiced with an adenovirus vector, e.g., an E1-, partial E3-, E4-deleted adenoviral vector may be used in the practice of the invention. Such vectors are safe as twenty-eight patients with advanced neovascular age-related macular degeneration (AMD) were given a single intravitreous injection of an E1-, partial E3-, E4-deleted adenoviral vector expressing human pigment epithelium-derived factor (AdPEDF.ll) (see, e.g., Campochiaro et al., Human Gene Therapy 17:167-176 (February 2006)); and previous adenovirus doses ranging from 10⁶ to 10^{9.5} particle units (PU) can be adapted to or employed in the practice of the instant invention (see, e.g., Campochiaro et al., Human Gene Therapy 17:167-176 (February 2006)). Adenoviral vector-mediated RNA transfer appears to be a viable approach for delivery of RNA(S). For adenoviral vector injections into a rat, 2×10⁹ infectious particles were injected in 3 ml of normal saline solution (NSS). This can be adapted to or extrapolated from in the practice of the present invention. For siRNA, a rat was injected into the great saphenous vein with 12.5 μg of a siRNA and a primate was injected injected into the great saphenous vein with 750 μg of a siRNA. This can be adapted to or extrapolated from in the practice of the present invention.

Accordingly, the invention contemplates amongst vector(s) useful in the practice of the invention: viral vectors, including retroviral vectors, lentiviral vectors, adenovirus vectors, or AAV vectors.

Several types of particle and nanoparticle delivery systems and/or formulations are known to be useful in a diverse spectrum of biomedical applications; and particle and nanoparticle delivery systems in the practice of the instant invention can be as in WO 2014/093622 (PCT/US13/74667). In general, a particle is defined as a small object that behaves as a whole unit with respect to its transport and properties. Particles are further classified according to diameter Coarse particles cover a range between 2,500 and 10,000 nanometers. Fine particles are sized between 100 and 2,500 nanometers. Ultrafine particles, or nanoparticles, are generally between 1 and 100 nanometers in size. The basis of the 100-nm limit is the fact that novel properties that differentiate particles from the bulk material typically develop at a critical length scale of under 100 nm. As used herein, a particle delivery system/formulation is defined as any biological delivery system/formulation which includes a particle in accordance with the present invention. A particle in accordance with the present invention is any entity having a greatest dimension (e.g. diameter) of less than 100 microns (μm). In some embodiments, inventive particles have a greatest dimension of less than 10 μm. In some embodiments, inventive particles have a greatest dimension of less than 2000 nanometers (nm). In some embodiments, inventive particles have a greatest dimension of less than 1000 nanometers (nm). In some embodiments, inventive particles have a greatest dimension of less than 900 nm, 800 nm, 700 nm, 600 nm, 500 nm, 400 nm, 300 nm, 200 nm, or 100 nm. Typically, inventive particles have a greatest dimension (e.g., diameter) of 500 nm or less. In some embodiments, inventive particles have a greatest dimension (e.g., diameter) of 250 nm or less. In some embodiments, inventive particles have a greatest dimension (e.g., diameter) of 200 nm or less. In some embodiments, inventive particles have a greatest dimension (e.g., diameter) of 150 nm or less. In some embodiments, inventive particles have a greatest dimension (e.g., diameter) of 100 nm or less. Smaller particles, e.g., having a greatest dimension of 50 nm or less are used in some embodiments of the invention. In some embodiments, inventive particles have a greatest dimension ranging between 25 nm and 200 nm. Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarisation interferometry and nuclear magnetic resonance (NMR). Characterization (dimension measurements) may be made as to native particles (i.e., preloading) or after loading of the cargo to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Particles delivery systems within the scope of the present invention may be provided in any form, including but not limited to solid, semi-solid, emulsion, or colloidal particles. As such any of the delivery systems described herein, including but not limited to, e.g., lipid-based systems, liposomes, micelles, microvesicles, exosomes, or gene gun may be provided as particle delivery systems within the scope of the present invention.

In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. Nanoparticles encompassed in the present invention may be provided in different forms, e.g., as solid nanoparticles (e.g., metal such as silver, gold, iron, titanium), non-metal, lipid-based solids, polymers), suspensions of nanoparticles, or combinations thereof. Metal, dielectric, and semiconductor nanoparticles may be prepared, as well as hybrid structures (e.g., core-shell nanoparticles). Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention.

Semi-solid and soft nanoparticles have been manufactured, and are within the scope of the present invention. A prototype nanoparticle of semi-solid nature is the liposome. Various types of liposome nanoparticles are currently used clinically as delivery systems for anticancer drugs and vaccines. Nanoparticles with one half hydrophilic and the other half hydrophobic are termed Janus particles and are particularly effective for stabilizing emulsions. They can self-assemble at water/oil interfaces and act as solid surfactants. Doses of about 5 mg/kg are contemplated, with single or multiple doses, depending on the target tissue. It is mentioned herein experiments involving mice involve 20 g mammals and that dosing can be scaled up to a 70 kg human. With regard to nanoparticles that can deliver RNA, see, e.g., Alabi et al., Proc Natl Acad Sci USA. 2013 Aug. 6; 110(32):12881-6; Zhang et al., Adv Mater. 2013 Sep. 6; 25(33):4641-5; Jiang et al., Nano Lett. 2013 Mar. 13; 13(3):1059-64; Karagiannis et al., ACS Nano. 2012 Oct. 23; 6(10):8484-7; Whitehead et al., ACS Nano. 2012 Aug. 28; 6(8):6922-9 and Lee et al., Nat Nanotechnol. 2012 Jun. 3; 7(6):389-93. Lipid Nanoparticles, Spherical Nucleic Acid (SNA^{T}M) constructs, nanoplexes and other nanoparticles (particularly gold nanoparticles) are also contemplate as a means for delivery A recent publication, entitled “In vivo endothelial siRNA delivery using polymeric nanoparticles with low molecular weight” by James E. Dahlman and Carmen Barnes et al. Nature Nanotechnology (2014) published online 11 May 2014, doi:10.1038/nnano.2014.84, incorporated herein in its entirety, showed that polymeric nanoparticles made of low-molecular-weight polyamines and lipids can deliver siRNA to endothelial cells with high efficiency, thereby facilitating the simultaneous silencing of multiple endothelial genes in vivo. The authors reported that unlike lipid or lipid-like nanoparticles, the nanoparticle formulation they used (termed 7C1), differed from traditional lipid-based nanoparticle formulations because it can deliver siRNA to lung endothelial cells at low doses without substantially reducing gene expression in pulmonary immune cells, hepatocytes or peritoneal immune cells.

Nucleic acids, amino acids and proteins: The invention uses nucleic acids to bind target DNA sequences. This is advantageous as nucleic acids are much easier and cheaper to produce than proteins, and the specificity can be varied according to the length of the stretch where homology is sought. Complex 3-D positioning of multiple fingers, for example is not required. The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridising to the reference sequence under highly stringent conditions. Generally, in order to maximize the hybridization rate, relatively low-stringency hybridization conditions are selected: about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately-stringent washing conditions are selected to be about 15 to 30° C. lower than the Tₘ. Highly permissive (very low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences. Preferred highly stringent conditions comprise incubation in 50% formamide, 5×SSC, and 1% SDS at 42° C., or incubation in 5×SSC and 1% SDS at 65° C., with wash in 0.2×SSC and 0.1% SDS at 65° C. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the dTALEs described herein have sequences that are at least 95% identical or share identity to the capping region amino acid sequences provided herein. Sequence homologies may be generated by any of a number of computer programs known in the art, for example BLAST or FASTA, etc. A suitable computer program for carrying out such an alignment is the GCG Wisconsin Bestfit package (University of Wisconsin, U.S.A; Devereux et al., 1984, Nucleic Acids Research 12:387). Examples of other software than may perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However it is preferred to use the GCG Bestfit program. Percentage (%) sequence homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues. Although this is a very simple and consistent method, it fails to take into consideration that, for example, in an otherwise identical pair of sequences, one insertion or deletion may cause the following amino acid residues to be put out of alignment, thus potentially resulting in a large reduction in % homology when a global alignment is performed. Consequently, most sequence comparison methods are designed to produce optimal alignments that take into consideration possible insertions and deletions without unduly penalizing the overall homology or identity score. This is achieved by inserting “gaps” in the sequence alignment to try to maximize local homology or identity. However, these more complex methods assign “gap penalties” to each gap that occurs in the alignment so that, for the same number of identical amino acids, a sequence alignment with as few gaps as possible—reflecting higher relatedness between the two compared sequences—may achieve a higher score than one with many gaps. “Affinity gap costs” are typically used that charge a relatively high cost for the existence of a gap and a smaller penalty for each subsequent residue in the gap. This is the most commonly used gap scoring system. High gap penalties may, of course, produce optimized alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension. Calculation of maximum % homology therefore first requires the production of an optimal alignment, taking into consideration gap penalties. A suitable computer program for carrying out such an alignment is the GCG Wisconsin Bestfit package (Devereux et al., 1984 Nuc. Acids Research 12 p387). Examples of other software than may perform sequence comparisons include, but are not limited to, the BLAST package (see Ausubel et al., 1999 Short Protocols in Molecular Biology, 4^{th} Ed.—Chapter 18), FASTA (Altschul et al., 1990 J. Mol. Biol. 403-410) and the GENEWORKS suite of comparison tools. Both BLAST and FASTA are available for offline and online searching (see Ausubel et al., 1999, Short Protocols in Molecular Biology, pages 7-58 to 7-60). However, for some applications, it is preferred to use the GCG Bestfit program. A new tool, called BLAST 2 Sequences is also available for comparing protein and nucleotide sequences (see FEMS Microbiol Lett. 1999 174(2): 247-50; FEMS Microbiol Lett. 1999 177(1): 187-8 and the website of the National Center for Biotechnology information at the website of the National Institutes for Health). Although the final % homology may be measured in terms of identity, the alignment process itself is typically not based on an all-or-nothing pair comparison. Instead, a scaled similarity score matrix is generally used that assigns scores to each pair-wise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table, if supplied (see user manual for further details). For some applications, it is preferred to use the public default values for the GCG package, or in the case of other software, the default matrix, such as BLOSUM62. Alternatively, percentage homologies may be calculated using the multiple alignment feature in DNASIS™ (Hitachi Software), based on an algorithm, analogous to CLUSTAL (Higgins D G & Sharp P M (1988), Gene 73(1), 237-244). Once the software has produced an optimal alignment, it is possible to calculate % homology, preferably % sequence identity. The software typically does this as part of the sequence comparison and generates a numerical result. The sequences may also have deletions, insertions or substitutions of amino acid residues which produce a silent change and result in a functionally equivalent substance. Deliberate amino acid substitutions may be made on the basis of similarity in amino acid properties (such as polarity, charge, solubility, hydrophobicity, hydrophilicity, and/or the amphipathic nature of the residues) and it is therefore useful to group amino acids together in functional groups. Amino acids may be grouped together based on the properties of their side chains alone. However, it is more useful to include mutation data as well. The sets of amino acids thus derived are likely to be conserved for structural reasons. These sets may be described in the form of a Venn diagram (Livingstone C. D. and Barton G. J. (1993) “Protein sequence alignments: a strategy for the hierarchical analysis of residue conservation” Comput. Appl. Biosci. 9: 745-756) (Taylor W. R. (1986) “The classification of amino acid conservation” J. Theor. Biol. 119; 205-218). Conservative substitutions may be made, for example according to the table below which describes a generally accepted Venn diagram grouping of amino acids.

| Set | | Sub-set | |
|---|---|---|---|
| Hydrophobic | F W Y H K M I L V A G C | Aromatic | F W Y H |
| | | Aliphatic | I L V |
| Polar | W Y H K R E D C S T N Q | Charged | H K R E D |
| | | Positively | H K R |
| | | Charged | |
| | | Negatively | E D |
| | | Charged | |
| Small | V C A G S P T N D | Tiny | A G S |

Embodiments of the invention include sequences (both polynucleotide or polypeptide) which may comprise homologous substitution (substitution and replacement are both used herein to mean the interchange of an existing amino acid residue or nucleotide, with an alternative residue or nucleotide) that may occur i.e., like-for-like substitution in the case of amino acids such as basic for basic, acidic for acidic, polar for polar, etc. Non-homologous substitution may also occur i.e., from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine. Variant amino acid sequences may include suitable spacer groups that may be inserted between any two amino acid residues of the sequence including alkyl groups such as methyl, ethyl or propyl groups in addition to amino acid spacers such as glycine or O-alanine residues. A further form of variation, which involves the presence of one or more amino acid residues in peptoid form, may be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR.

In certain aspects the invention involves vectors. A used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid of the invention in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell). With regards to recombination and cloning methods, mention is made of U.S. patent application Ser. No. 10/815,730, published Sep. 2, 2004 as US 2004-0171156 A1, the contents of which are herein incorporated by reference in their entirety.

In practicing any of the methods disclosed herein, a suitable vector can be introduced to a cell or an embryo via one or more methods known in the art, including without limitation, microinjection, electroporation, sonoporation, biolistics, calcium phosphate-mediated transfection, cationic transfection, liposome transfection, dendrimer transfection, heat shock transfection, nucleofection transfection, magnetofection, lipofection, impalefection, optical transfection, proprietary agent-enhanced uptake of nucleic acids, and delivery via liposomes, immunoliposomes, virosomes, or artificial virions. In some methods, the vector is introduced into an embryo by microinjection. The vector or vectors may be microinjected into the nucleus or the cytoplasm of the embryo. In some methods, the vector or vectors may be introduced into a cell by nucleofection.

The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g. 1, 2, 3, 4, 5, or more pol Ill promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pot III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EFla promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89). In some embodiments, a vector is a yeast expression vector. Examples of vectors for expression in yeast Saccharomyces cerivisae include pYepSecl (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.). In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In some embodiments, a vector is capable of driving expression of one or more sequences in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. FMBO J. 8: 729-733) and immunoglobulins (Baneiji, et al., 1983. Cell 33: 729-740; Queen and Baltimore, 1983. Cell 33: 741-748), neuron-specific promoters (e.g., the neurofilament promoter; Byrne and Ruddle, 1989. Proc. Nal. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments of the invention may relate to the use of viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Results

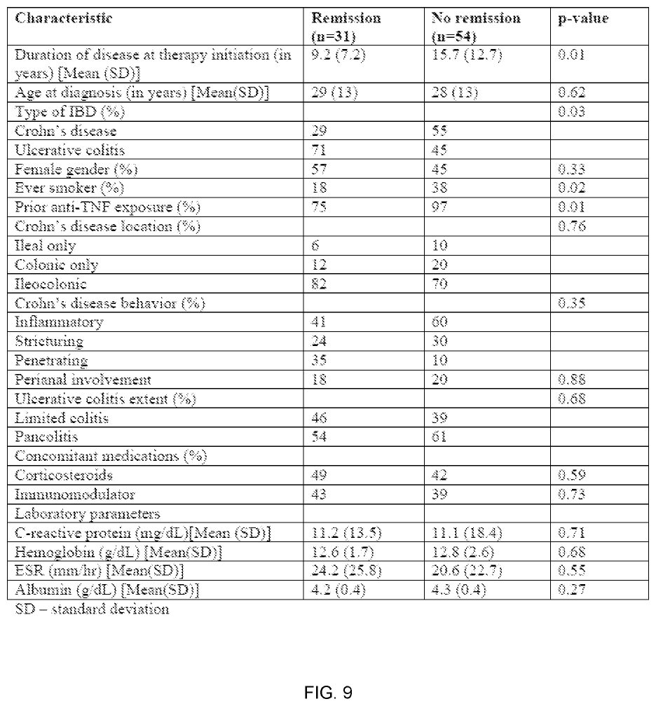

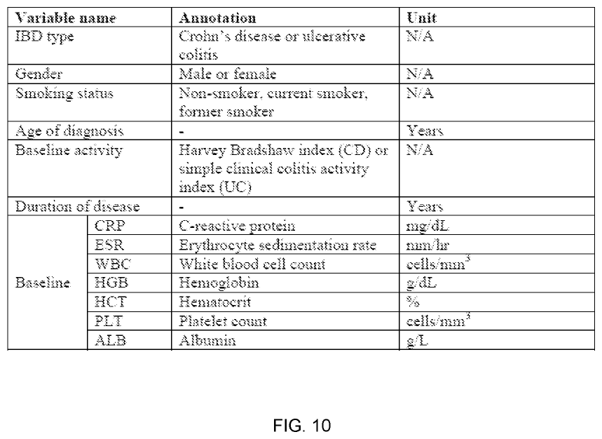

Study population. The study included 85 patients with IBD (43 UC, 42 CD) with a mean disease duration of 13 years at the start of therapy. Just under half of the patients were on concomitant therapy with immunomodulators (42%). Most had previously failed an anti-TNF agent. The mean HBI and SCCAI at baseline were 6 and 5.9 respectively with a mean CRP of 13.2 mg/L (range 0.1-140). At week 14, 31 patients met Applicants' primary outcome of clinical remission. At week 54 (n=71), 35% of patients remained in remission. Patients who attained remission were likely to have had disease for a shorter duration, more likely to have a diagnosis of CD and less likely to have had prior anti-TNF exposure (p<0.05 for all) (FIG. 9).

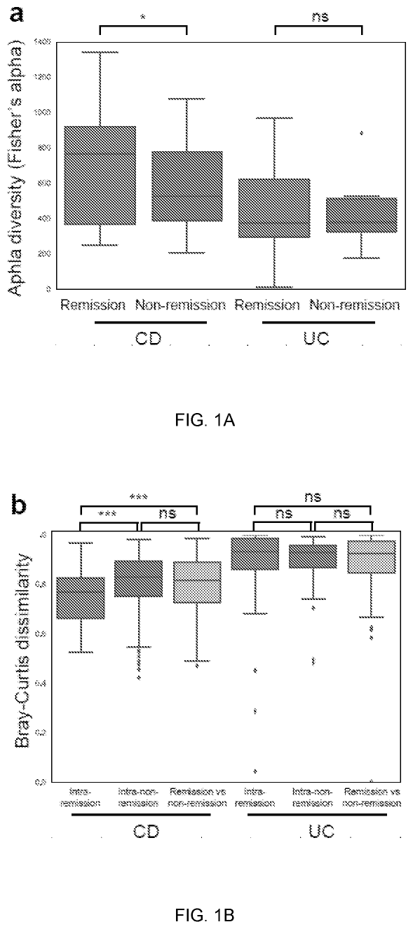

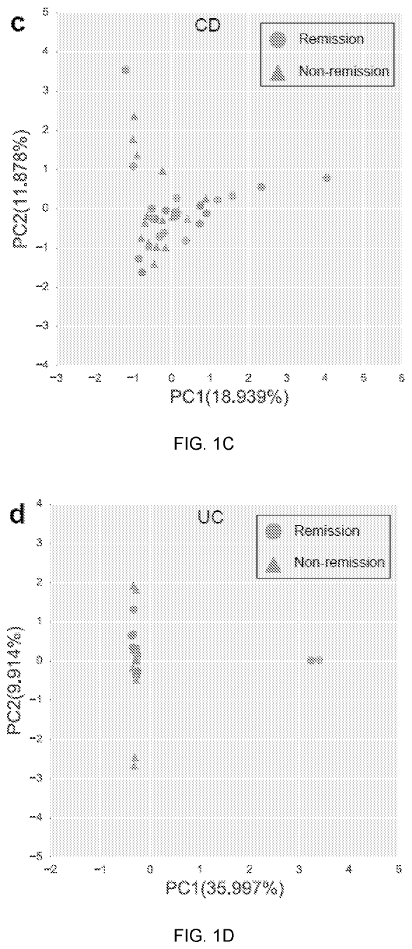

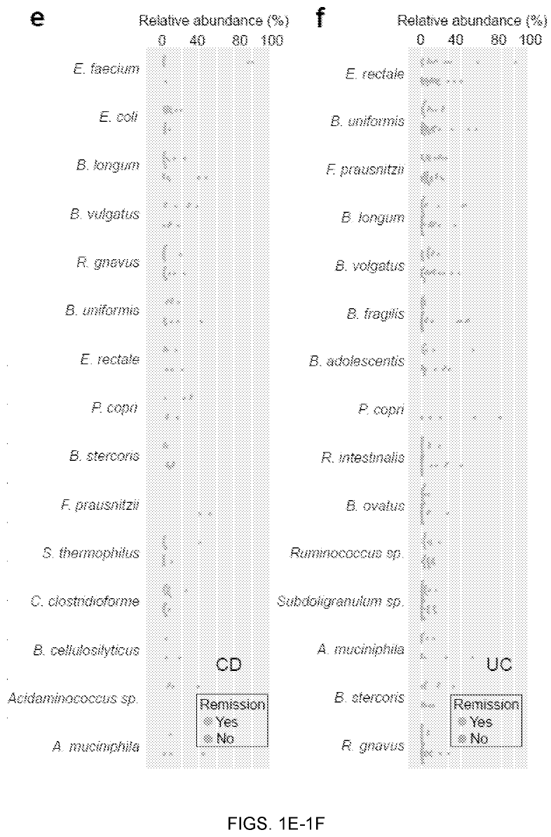

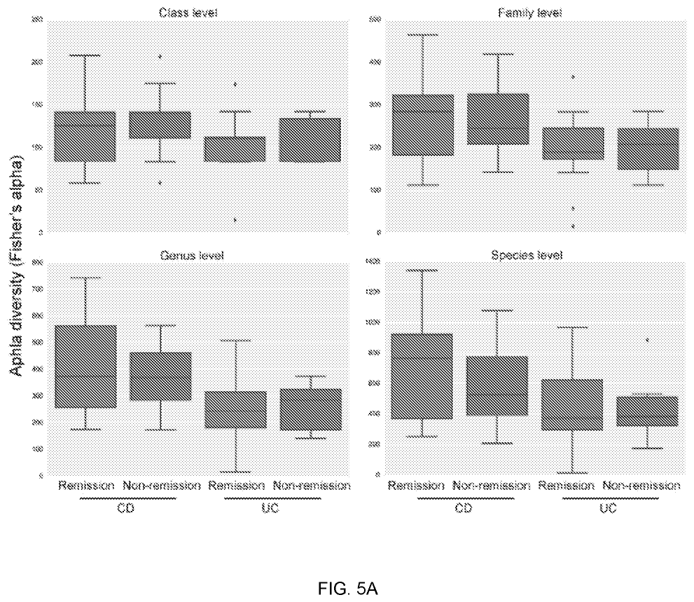

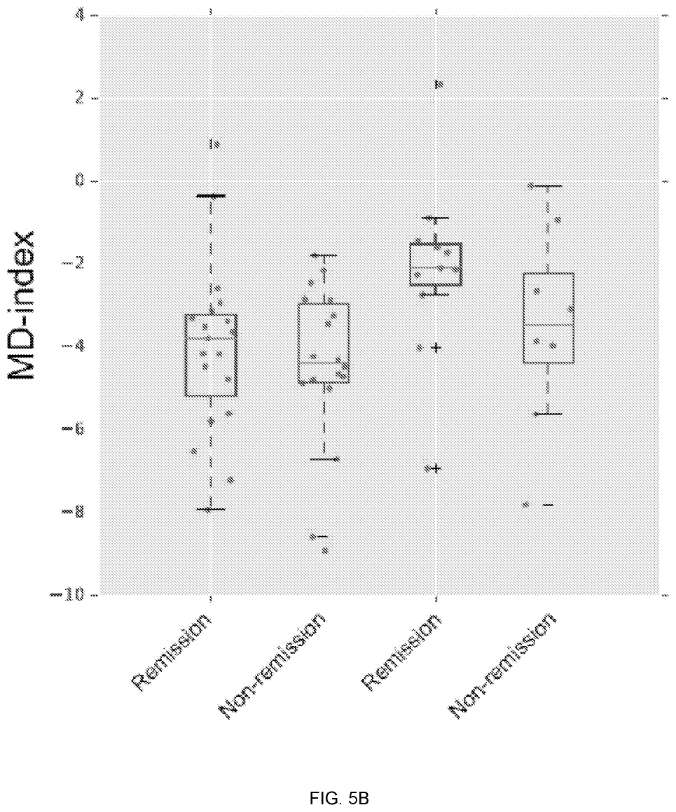

Baseline metagenomic composition and remission at week 14. Community alpha-diversity at baseline was significantly higher in CD patients who achieved remission at week 14 (q<0.1, student's t-test; FIG. 1a). This did not achieve statistical significance in UC though Applicants noted a wider range of baseline community diversity for those in remission (p=0.031, F test; FIG. 1a). This effect was only observed at the species level suggesting that fine taxonomic differences differentiate remission and non-remission groups (FIG. 5A). For CD patients, beta-diversity measured was lower at baseline within the remission group compared with the non-remission group (Figure ib). There was no difference in the microbiome dysbiosis index between remitters and non-remitters either in CD or UC (FIG. 5B).

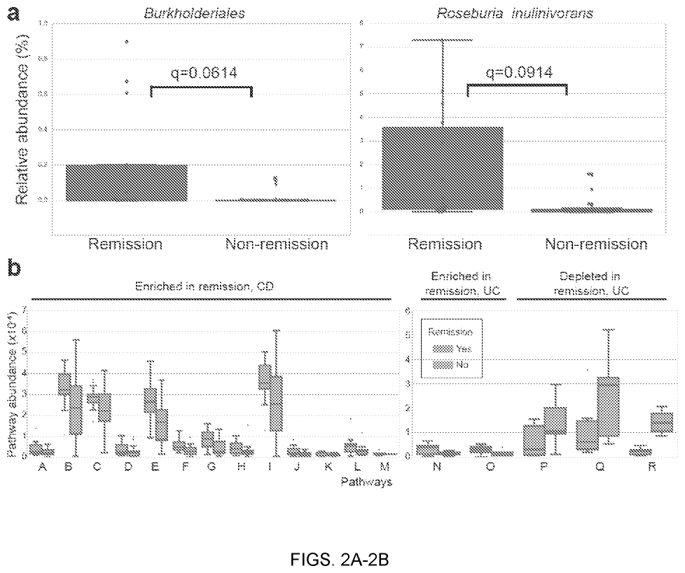

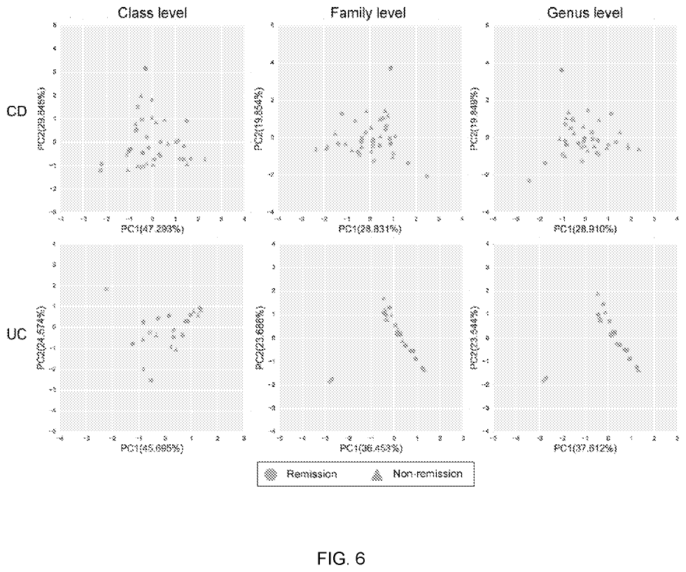

Principle component analysis (PCoA) did not differentiate remitters from non-remitters across different taxonomic ranks (FIGS. 1c-d, FIG. 6), possibly due to similar baseline relative abundance of the top 15 most abundant species among remitters and non-remitters (FIG. 1e-f). However, two species demonstrated a statistically significant difference in relative abundance at baseline between week 14 remitters and non-remitters. Both Roseburia inulinivoranrs and a Burkholderiales species were significantly more abundant at baseline among CD patients achieving week 14 remission compared to non-remitters (q=0.0914 for R. inulinivorans, q=0.0614 for Burkholderiales sp.; FIG. 2a).

Thirteen pathways were significantly enriched (q<0.1) in baseline samples from the CD patients achieving remission compared to non-remitters including branched chain amino acid (BCAA) biosynthesis pathways involved in biosynthesis of L-citrulline (log 2 fold difference: 0.812, q=0.0957), L-isoleucine from threonine (log 2 fold difference: 0.482, q=0.02), and arginine and polyamine (log 2 fold difference 1.073, q=0.0828) (FIG. 2b). Two and three pathways were respectively significantly enriched and depleted among UC patients achieving remission (q<0.1; FIG. 2b).

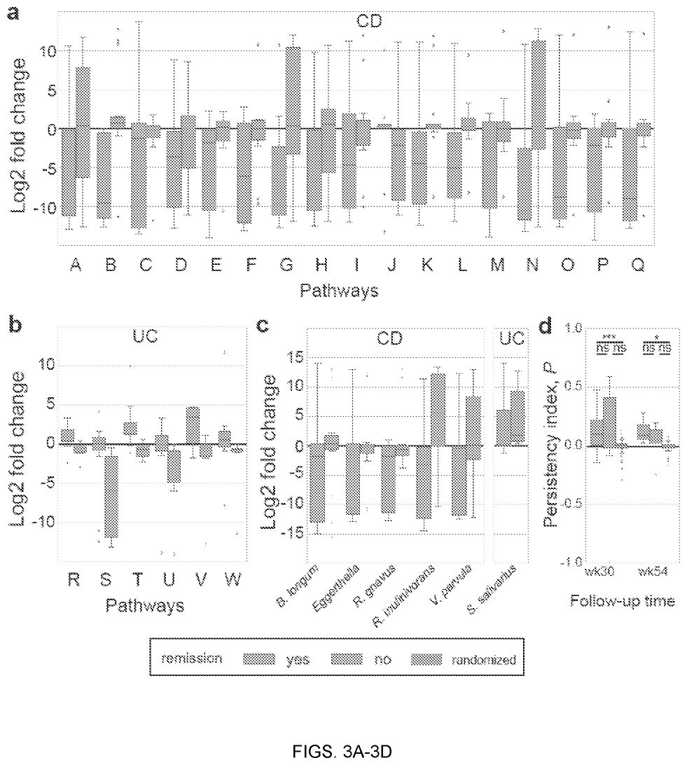

Longitudinal trajectory of the microbiome. In CD, only five taxa were significantly different in relative abundance between baseline and follow-up (24 CD patients with paired samples; 10 achieving remission). These were Bfidobacterium longum, Eggerthella, Ruminococcus gnavus, Roseburia inulinivorans, and Veillonella parvula (FIG. 3b). All these taxa decreased in relative abundance in patients achieving remission. In UC (17 patients with paired samples; 11 achieving remission), only one taxon, Strepotococcus salivarium, significantly changed in relative abundance with an increase in abundance in patients not achieving remission (FIG. 3b).

In contrast to these few changes in microbial composition, there were significantly greater metagenomic alterations in microbial function. In CD, 17 pathways were significantly reduced on follow-up at week 14 compared to baseline, of which 15 were noted only in patients achieving remission (FIG. 3a). These included a decrease in several tricarboxylic acid cyclic (TC) pathways (I and V types) and nicotinamide adenine dinucleotide (NAD) salvage pathway, suggesting decreased oxidative stress in patients achieving remission. O-antigen building blocks biosynthesis in E. coli was also decreased but not accompanied by a corresponding reduction in abundance of E. coli. In patients not achieving remission, only two pathways—L-arginine biosynthesis via N-acetyl-L-citrulline pathway and tetrapyrrole biosynthesis from glutamate pathway—were decreased by week 14. The changes were less striking in UC. Three pathways—polyamine biosynthesis, non-oxidative pentose phosphate pathway, and sucrose degradation—increased in relative abundance among patients achieving remission (FIG. 3a). In contrast, gluconeogenesis, uridine monophosphytate (UMP) biosynthesis, and putrescine biosynthesis decreased in relative abundance among those not achieving remission (FIG. 3a).

Finally, Applicants examined if there was a difference in the direction of change for taxa or pathways between those achieving remission and those not. The only significantly different species in CD was Roseburia inulinivorans which decreased in abundance in those achieving remission while increasing in those who had not (q=0.013; Benjamini-Hochberg adjusted χ² test). Hexitol degradation and glycolysis pathways also demonstrated different directions of change in CD patients achieving remission compared to those not. In UC, while no significant differential changes in taxa were noted, palmitate and stearate biosynthesis pathways increased in relative abundance in patients achieving remission and decreased in those not achieving remission (q=0.087 and 0.045, respectively; Benjamini-Hochberg adjusted χ² test).

Persistence of changes in the microbiome at 1 year. Eight patients (3 CD; 5 UC) provided stool samples at baseline, weeks 14, 30, and 54 while thirteen patients (5 CD; 8 UC) had samples available at baseline, weeks 14 and 54. Persistency of treatment effect was observable at both week 30 and week 54 for among both the remission and non-remission groups though only the changes in the remission group demonstrated a statistical difference compared to random sampling (FIG. 3d). Specifically, patients achieving remission at week 14 demonstrated highly significant persistency in the microbial composition at week 30 (P=0.00039) and a weaker effect at week 54 (P=0.019), suggesting that attainment of remission at week 14 is associated with durable changes in the microbiome.

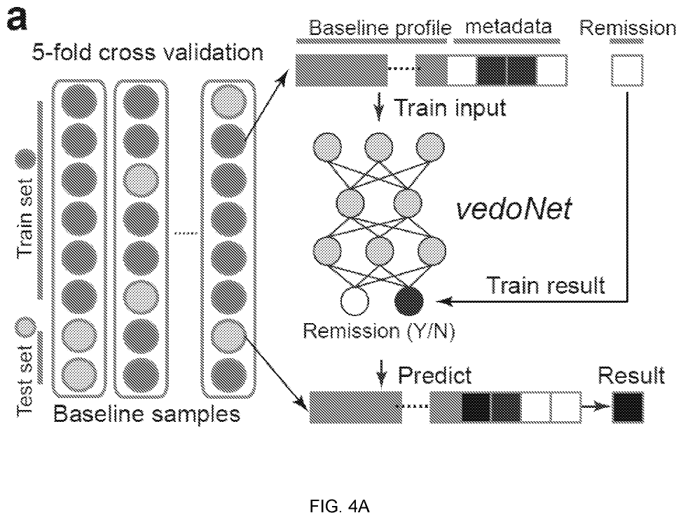

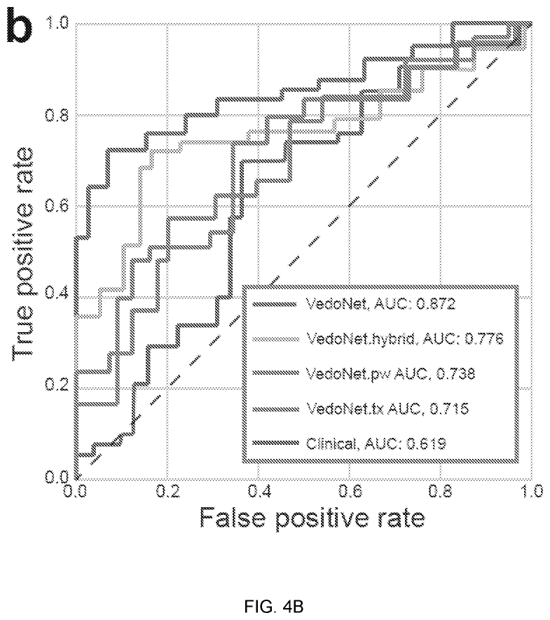

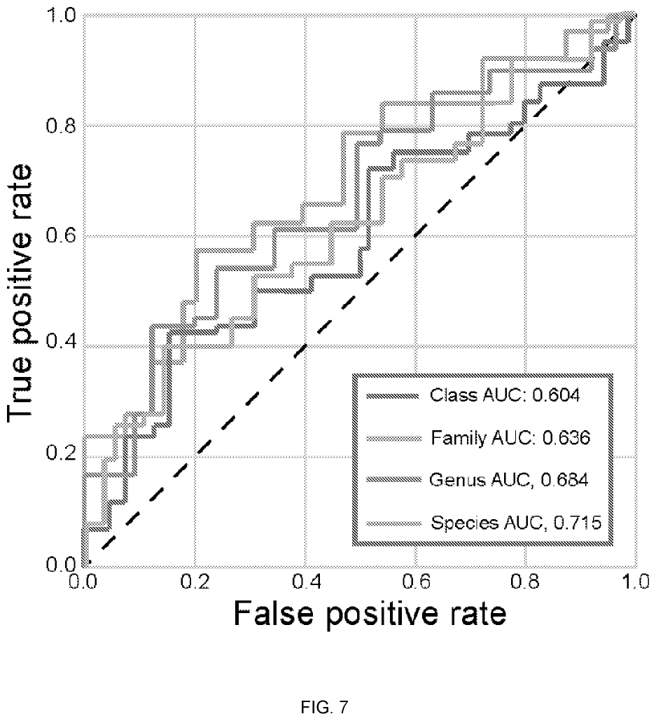

Neural network algorithms (vedoNet) to predict treatment response. Several different neural network models were evaluated to predict clinical remission at week 14 (FIGS. 4a-b, see Methods for model details). Baseline clinical data alone was insufficient in predicting remission at week 14 (AUC 0.619). In contrast, use of available microbial taxa (vedoNet.tx; AUC 0.715) and pathways (vedoNet.pw, AUC 0.738) resulted in improved predictive ability (FIG. 4b).Taxonomic profiles at the level of genus, family, or class performed less well than information at the species level (FIG. 7). A model incorporating clinical data, taxonomy, and pathway relative abundance without any pre-selection of variables performed better than each individual model (vedoNet.hybrid, AUC 0.776) (FIG. 4b). Finally, a manually curated list of 40 microbiome variables (Methods, FIG. 9) provided the highest classifying power (AUC=0.872; FIG. 4b), successfully achieving >80% true positive discovery rate with a less than 25% false negative discovery rate. For other models to achieve the same true positive rate, the false negative rates were over 50% (vedoNet.tx and vedoNet.pw) or 60% (clinical data and vedoNet.hybrid). vedoNet was implemented in Python with Keras library; the codes, models, and tutorials can be found at www.bitbucket.org/luo-chengwei/vedoNet. Applicants repeated the analysis stratifying by type of IBD at baseline, allowing for incorporation of disease-specific phenotypic information. This did not significant improvement the predictive value of the vedoNet model for either CD (AUC 0.881) or UC (AUC 0.853). In analysis stratified by MD-index (above or below median), among high MD-index subjects, vedoNet's sensitivity was 0.75 and specificity was 0.769; while in low MD-index subjects, vedoNet's sensitivity and specificity were 0.818 and 0.85 respectively.

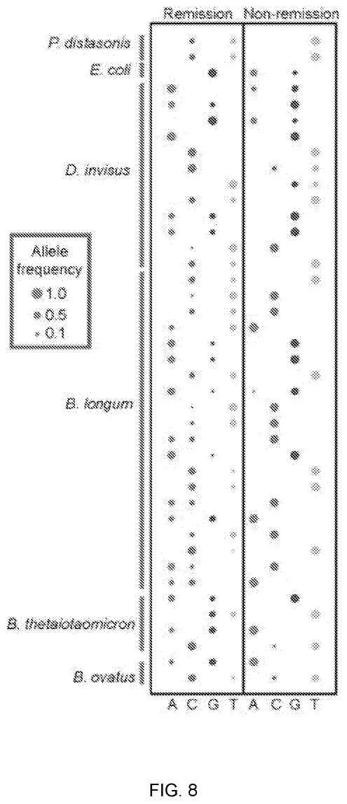

Strain-level analysis. To investigate if the strain variability can play a role in determining the outcome of the treatment, Applicants employed a strain-level resolution approach to identify any strain-specific signal showed significant association with outcome. Applicants sought to focus on pathways that differentiated remission group from non-remission group, since differences in genes within these pathways might offer essential information in determining treatment. Applicants found that among CD baseline samples, those who entered remission at week 14 possessed a cluster of unique SNPs (FDR<0.1) located in L-arginine biosynthesis pathways. Such SNPs are predominantly contributed by Bifidobacterium longum (21/41=51.22% of total unique SNPs) and Dialister invisus (11/41=26.83% of total unique SNPs); among UC baseline samples, those who entered remission at week 14 possessed a more diversified group-specific SNP profile that spread among UMP biosynthesis pathway and pentose phosphate pathway (FIG. 8). The major differentiating species contributing to the stratifying SNPs include Bifidobacterium longum, Ruminococcus torques, and E. coli.

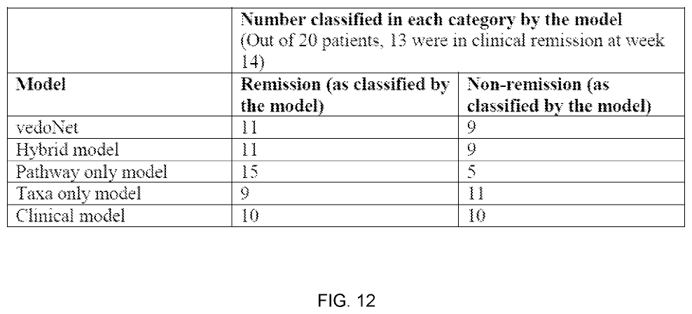

Validation in an anti-TNF cohort. Twenty patients (14 CD, 6 UC) were included in the anti-TNF validation cohort with a mean disease duration of 7 years. The baseline C-reactive protein levels were 16.9 mg/dL and mean HBI and SCCAI were 5 and 6 respectively. At week 14, 13 patients (65%) achieved clinical remission. FIG. 12 presents the results of the various classification algorithms in classifying remitters and non-remitters. vedoNet was able to accurately identify 11 out of the 13 patients achieving remission and was superior to models utilizing clinical data (10/13) or microbial taxa alone (9/13).

### Example 2: Discussion

The gut microbiome is a key determinant of initiation and propagation of luminal inflammation in IBD (Becker et al., 2015; Forbes et al., 2016; Gevers et al., 2014; Knights et al., 2013; Kostic et al., 2014). Here, Applicants describe the microbial composition and structure from a large cohort of IBD patients initiating vedolizumab therapy (Shelton et al., 2015). Applicants demonstrate associations between baseline taxonomic composition and functional pathway abundance and clinical remission at 14 weeks and demonstrate the utility of predictive models incorporating both clinical and microbiome data in predicting clinical remission. Applicants also hypothesize that trajectory of early changes in the microbiome may be a marker of response to treatment in IBD.

There have been few studies of longitudinal changes in the gut microbiome with drug treatment in IBD. Shaw et al. characterized 19 children with CD and 4 with UC, showing dysbiosis at baseline that correlated with luminal inflammatory burden (Shaw et al., 2016). An improvement in fecal diversity was seen with clinical response in UC but not CD. In Applicants' study, a more diverse microbial composition at baseline predicted week 14 clinical remission. A less diverse microbiome has been consistently linked to the development of IBD; factors such as antibiotics that reduce gut diversity increase risk of IBD (Becker et al., 2015; Forbes et al., 2016; Gevers et al., 2014; Knights et al., 2013; Kostic et al., 2014; Lewis et al., 2015; Singh et al., 2009; Ungaro et al., 2014). Thus, a more diverse microbiome at baseline may reflect prevalent microbes and/or metabolites with anti-inflammatory effect on colonic inflammation and a less disrupted mucosal barrier, leading to greater treatment response. Restoration of gut diversity with has been reported previously with anti-TNF therapy (Lewis et al., 2015; Shaw et al., 2016) though a more diverse microbiome has not been previously shown to be predictive of treatment response. One could hypothesize that this difference may be due to the systemic effect of anti-TNF therapy compared to the inhibition of gut-specific leukocyte trafficking by vedolizumab. Microbiome derived signals maybe more relevant to response to agents that block T cell traffic as compared to antibodies that neutralize specific cytokines. The complexity in predicting treatment response using gut microbial structure is highlighted by poor separation between the remitters and non-remitters on simple PCoA, consistent with a few previous studies (Shaw et al., 2016). Applicants' more adaptive and informative neural network based approach performed significantly better with an AUC of 0.87 and suggested added and complementary value to both clinical and microbial parameters.

Taxonomically, the relative abundance of R. inulinivorans and Burkholderiales at baseline was predictive of week 14 remission. R. inulinivorans is a relatively low abundance gram-positive organism, certain strains of which encode genes for pro-inflammatory flagellin proteins that stimulate interleukin-8 production (Neville et al., 2013). R inulinivorans also produces butyrate and propionate both of which have anti-inflammatory effects through a variety of mechanisms including reinforcing the integrity of the colonic epithelial barrier, reducing oxidative stress, and decreasing inflammation through inhibition of nuclear factor κB (NF-κB ) activation by histone deacetylation (Canani et al., 2011; Hamer et al., 2008; Inan et al., 2000). Butyrate also inhibits inflammation through inhibition of the IFNγ/STAT1 signaling pathways associated with chronic inflammation and enhances apoptosis of colonic T-cells (Hamer et al., 2008; Zimmerman et al., 2012).

In contrast to the relatively few changes between remitters and non-remitters at the species or genus level, differences in functional pathways were more striking. Pathways related to BCAA biosynthesis including citrulline, isoleucine, arginine and polyamine were enriched at baseline in CD patients who achieved week 14 remission. BCAA may reduce colonic inflammation through a variety of mechanisms. Arginine and isoleucine supplementation results in upregulation of human beta defensin 1 (hBD-1) in colon cells; reduced beta-defensin expression is associated with colonic inflammation in IBD (Ramasundara et al., 2009). In a C57BL/J6 mouse dextran sodium sulfate (DSS) colitis model, arginine supplementation reduced intestinal inflammation and cytokine production (Coburn et al., 2012). Arginine is also a precursor for nitric oxide (NO) and endothelial NO is important for maintenance of intestinal perfusion and barrier integrity while NO produced by the inducible nitric-oxide synthase has direct anti-bacterial activity and is an important regulator of host defense (Kolios et al., 2004). NO may also reduce damage from oxidative stress and through inhibition of NF-kB translocation (Kolios et al., 2004). Glycosaminoglycan (GAG) degradation pathways were also enriched in those achieving remission. In mice models, intestinal flora mediated degradation of GAG resulted in metabolites with a cytotoxic effect on intestinal epithelium; inhibition of this degradation with antibiotics ameliorated colitis (Lee et al., 2009). Week 14 remission was associated with a reduction in several functional pathways up regulated at baseline. For example, the NAD salvage pathway decreased by week 14 among those achieving remission suggesting that clinical improvement was associated with a reduction in luminal oxidative stress. These discoveries together suggest that functional rather than taxonomic differences may be important determinants of treatment outcome.

Interestingly, Applicants also observed that responders at week 14 demonstrated greater persistence of their microbial changes at 1 year compared to non-responders suggesting that early changes in the microbiome could be an indicator of clinical response. Similar clinical observations have been noted in CD and UC. In the ACT trial of infliximab in UC, endoscopic response by week 8 was associated with a lower rate of colectomy at week 54(Colombel et al., 2011). In parallel, early reduction in fecal calprotectin has been associated with improved long-term outcomes in patients with IBD (Pavlidis et al., 2016). Thus, early microbiome changes may be an added marker of sensitivity to treatment and initial response.

There are several implications to Applicants' findings. Applicants' study demonstrates the ability to predict response to anti-integrin treatment using the gut microbiome, highlighting the role not just of microbial taxonomy but more importantly functional pathways that may be relevant to treatment. Similar analyses for drugs with different mechanisms of action may offer the ability to a priori select agents with higher likelihood of response based on gut microbial composition. Advances in technology increasingly allow rapid sequencing of the microbiome through methods such as paper-based assays (Pardee et al., 2014), and one could envision such methods being incorporated into routine clinical care. The persistence of microbial changes in those achieving remission at week 14 further highlights the importance of early clinical response in predicting long-term outcome with treatment, and potentially a mechanism thereof. Identification of predictive microbial signals could also allow for refinement of novel probiotics that may deliver specific anti-inflammatory taxa or strains, or stimulate anti-inflammatory metabolic pathways that may be of benefit in ameliorating gut inflammation.

Applicants readily acknowledge several limitations to the study. This was a single center cohort of predominantly refractory patients. Remission relied on clinical indicators rather than biochemical, fecal, or endoscopic outcomes. Few patients provided stools at each of the time points through 1 year, limiting Applicants' statistical power. Diet was not routinely assessed in all patients, and consequently its effect on the gut microbiome cannot be excluded. While Applicants performed validation in a model in an independent cohort of 20 patients initiating anti-TNF therapy, Applicants acknowledge that more robust examination in larger cohorts is essential prior to application to clinical practice. Further experimental studies are important to determine the full mechanistic implications of the metagenomic pathways and bacteria identified and how they may be harnessed to improve response to existing therapy in IBD.

In conclusion, Applicants describe associations between gut microbial taxonomic composition and function and response to anti-integrin therapy in CD and UC. Early clinical remission could be predicted by microbial functional composition at baseline with a weaker influence at the level of the species or genus. The association between abundance of butyrate producing bacteria and enrichment of branched chain amino acid biosynthesis pathways at baseline in remitters, and reduction in oxidative stress pathways with therapy response provides support for an important role of these pathways in the propagation and resolution of intestinal inflammation. The pathways and microbes thus identified could potentially serve as targets for newer therapies and shed further light on the pathogenesis and progression of these complex diseases.

### Example 3: Star Methods

Vedolizumab cohort and outcomes. This study was nested within a longitudinal prospective IBD cohort at Massachusetts General Hospital (Prospective Registry of IBD Study at MGH (PRISM)). Details of this cohort have been published previously (Ananthakrishnan et al., 2014; Shelton et al., 2015). In brief, the PRISM registry is open to all adult patients with IBD seeking care at the MGH Crohn's and Colitis center. This nested study was a prospective inception cohort of patients initiating vedolizumab for refractory luminal CD or UC, often in the setting of prior anti-tumor necrosis factor α (anti-TNF) failure. Characteristics of the included patients initiating vedolizumab therapy is presented in FIG. 9. All patients initiating vedolizumab as part of their routine clinical care were eligible for inclusion without an a priori fixed sample size for recruitment. Patients with an ileostomy or J-pouch were excluded as disease activity scores could not be reliably calculated. Most patients had failed more than one anti-TNF therapy previously. Patients received intravenous vedolizumab 300 mg at weeks 0, 2, 6, and every 8 weeks thereafter. At weeks 0 (baseline), 6, 14, 30, and 54, patients provided stool for metagenomic sequencing. At each infusion, disease activity was assessed using the Harvey Bradshaw index for CD (Harvey and Bradshaw, 1980) and simple clinical colitis activity index for UC (Walmsley et al., 1998). Hemoglobin, serum albumin, C-reactive protein, erythrocyte sedimentation rate, white blood cell and platelet count were obtained at each infusion. Applicants' primary study outcome was clinical remission at week 14, defined as HBI <4 or SCCAI<2. A reduction in either the HBI or SCCAI by ≥3 points indicated clinical response, consistent with the cut-offs used in clinical trials (Harvey and Bradshaw, 1980; Walmsley et al., 1998).

Validation cohort. External validation of the results of Applicants' predictive model was performed in an independent cohort of 20 patients with moderate-to-severe CD or UC initiating therapy with an anti-TNF biologic therapy (infliximab or adalimumab). Similar to the vedolizumab cohort, disease activity using the HBI or SCCAI was collected along with stool for metagenomic sequencing at baseline and at week 14. Applicants examined the ability of the final predictive model developed in the vedolizumab cohort to classify anti-TNF remitters and non-remitters at week 14.

Microbiome community profiling and sequencing. RNA and DNA purification from stool aliquots was performed according to protocols optimized in the Human Microbiome Project (Group et al., 2009; Integrative, 2014). In brief, participating patients provided stool in storage tubes containing RNA later. Stool samples were stored at 4 C for for less than 24 hours and then stored at −80 C until DNA extraction. Genomic DNA extraction from stool was performed using the Qiagen AllPrep MiniKit (Valencia, Calif., USA) as per manufacturer's instructions. Illumina based DNA shotgun sequencing was performed at the Broad Institute (Cambridge, Mass.) to characterize rare taxa and understand relationships between community membership and community function. Metagenomic analysis. Metagenomic reads were quality trimmed using trimmomatic v0.36 with default settings, retaining post-trimming reads with both ends longer than 60 bp (Bolger et al., 2014). Samples were minimized for reads originating in human genomes using BMTagger (ftp-//ftp.ncbi.nlm.nih.gov/pub/agarwala/bmtagyer., 7 Mar. 2011, version 3.101). MetaPhlan2(Truong et al., 2015) was employed to taxonomically profile each sample using default settings with Bowtie v2.2.4 as search engine (Langmead and Salzberg, 2012). Pathway relative abundance of each sample was quantified by HUMaN v2.0(Abubucker et al., 2012) using DIAMOND (Buchfink et al., 2015) with package-shipped ChocoPhlAn and EC-filtered UniRef90 databases. Fisher's Alpha was calculated for the taxonomic profiles at phylum, class, order, family, genus, and species levels for each sample using normalized read count from MetaPhlAn2's marker gene mapping. Bray-Curtis dissimilarity (BCD) was calculated in intra-group and inter-group fashions on the same taxonomic ranks. The top most abundant taxonomic groups and pathways were selected by the median relative abundance across all samples. Student's i-test was carried out to test if any taxonomic group, pathway, or diversity metrics at baseline significantly differed between those achieving remission at week 14 and those who did not; p-values were corrected for multiple testing using the Benjamini-Hochberg procedure. The F test was used to examine if the two normal distributions significantly differed in their variances. Applicants also calculated a microbiome dysbiotic index (MD-index) as the logarithm of the ratio between the relative abundance sum of IBD-increased taxonomic groups and the relative abundance sum of the IBD-decreased taxonomic groups defined by Gevers et al. Differences in the MD-index between remitters and non-remitters were compared.

Analysis of longitudinal trajectory of the gut microbiome. For IBD patients with both baseline and week 14 stool samples available, Applicants calculated the log 2 fold change (FC) in taxa and pathway relative abundance. The change was defined as significant if a taxon or pathway experienced consistent ≥1.5 FC (log 2FC values are ±0.58) in over 80% of the samples. For subjects with follow-up samples available at later time points (weeks 30 and 54), Applicants designed a persistent index to measure the degree of persistency of the effect of treatment on taxa or pathways. This index was defined as the difference in the degree of the later follow-up sample mimicking the week 14 sample accounting for baseline differences. Formally, the index, P, was defined as:

P=[BCD(f,b)−BCD(f,k)]/BCD(k,b),

where BCD(x, y) represents the Bray-Curtis dissimilarity between sample x and y, and b, k, and f defined the baseline, week 14, and later follow-up (week 30 or week 54) samples. If sample f was identical to sample k representing maximum persistency, then P=1. If sample f was identical to sample b representing no persistency, then P=−1. A randomized profile was generated by decoupling the taxa and relative abundance and re-associating them at random. P was calculated for the randomized samples and independent student's t-test was applied to analyze differences between random and observed profiles.

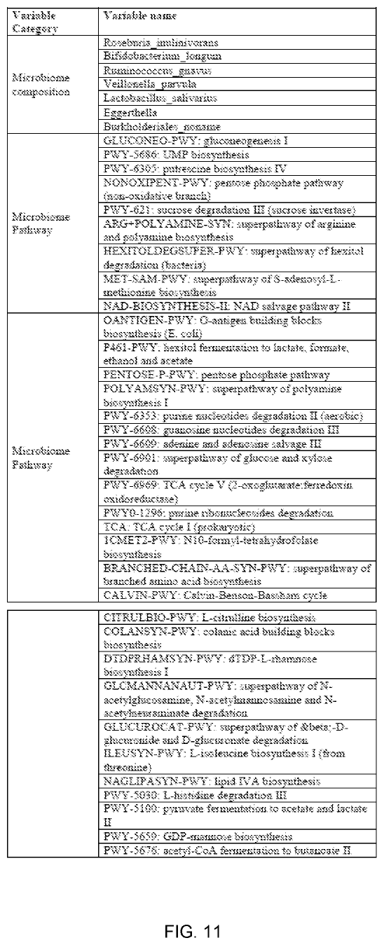

Neural network predictor, vedoNet. A neural network structure-based predictor was constructed using baseline information to predict remission at week 14. This consisted of an input layer, a convolution layer with a softmax dropout layer, a rectified linear unit (ReLU), and an output unit to classify if the input data could predict week 14 remission. Random parameters drawn from standard norm ˜N(0,1) were assigned to the initial neural network, and the network was trained and tested on 5-fold cross validation. The input variables including both microbiome data as well as clinical information including type of IBD, age at diagnosis, gender, smoking history, baseline disease activity, and laboratory parameters (CRP, WBC count, ESR, platelet count, hemoglobin, and albumin) (FIG. 10). Different microbiome models were tested and compared, including purely taxon profiles-based (vedoNet.tx), pathway-based (vedoNet.pw), as well as mixture of both, with knowledge-guided input variable selection (vedoNet). For vedoNet.tx, MetaPhlan v2.0 profiles at species, genus, and class levels were respectively employed in model construction; for vedoNet.pw, normalized HUMANn2 output pathway profiles were used in model construction. Lastly, for vedoNet, Applicants selected the relative abundance of Roseburia inulinivorans, Burkholderiales, Eggerthella, Bifidobaterium longum, Ruminococcus gnavus, Veillonela parvida, Lactobacillus salivarius, and the relative abundance of pathways shown in FIG. 11 as input for model building. These phyla were selected based on the fold change difference between baseline and week 14 among the remission and non-remission groups. The number of neural units for the input layers varied to accommodate the different input vector lengths in each respective model.

The area under curve (AUC) of the receiver operating characteristic (ROC) curve served as the main indicator of vedoNet's performance. Subjects with complete follow up information at week 14 and a stool sample at baseline were divided into five batches at random for the 5-fold cross validation procedure. One-hundred such combinations were generated to train and test models. The training process deployed stochastic gradient descent (SGD) using categorical cross entropy as cost function. The model structure with the highest AUC was selected and re-trained in 1,000 iterations using SGD and calibrated with all samples.

Strain level analysis. To obtain strain-level resolution on the differentiating pathways, Applicants mapped every baseline sample's reads onto the reference genes using Bowtie2 (Langmead and Salzberg, 2012) and then piled up the reads using SAMTools (Li et al., 2009) mpileup function with default settings, controlling read mapping quality to be no less than 15. Applicants first called SNPs with reference-free, aggregated allele frequencies; positions with higher than 10× relative abundance in both sample groups (remission and non-remission) and minor allele with at least 2× coverage and frequency >0.1 were identified as SNPs. Based on week 14 followup remission status, Applicants calculated the likelihood that the two groups allele frequencies were drawn from different background. To quantify the SNPs' uniqueness in differentiating the two groups, Chi-2 test was carried out and further corrected for FDR using Benjamini-Hochberg approach. SNPs sites with q<0.1 were selected as unique SNP sites.

Data and Software availability. The data from the study are available at https://www.ncbi.nlm.nih gov/sra/.

### REFERENCES

Abubucker, S., Segata, N., Goll, J., Schubert, A. M., Izard, J., Cantarel, B. L., Rodriguez-Mueller, B., Zucker, J., Thiagarajan, M., Henrissat, B., et al. (2012). Metabolic reconstruction for metagenomic data and its application to the human microbiome. PLoS Comput Biol 8, e1002358.
Ananthakrishnan, A. N., Huang, H., Nguyen, D. D., Sauk, J., Yajnik, V., and Xavier, R. J. (2014). Differential effect of genetic burden on disease phenotypes in Crohn's disease and ulcerative colitis: analysis of a North American cohort. Am J Gastroenterol 109, 395-400.
Arijs, I., Li, K., Toedter, G., Quintens, R., Van Lommel, L., Van Steen, K., Leemans, P., De Hertogh, G., Lemaire, K., Ferrante, M., et al. (2009). Mucosal gene signatures to predict response to infliximab in patients with ulcerative colitis. Gut 58, 1612-1619.
Baumgart, D. C., and Sandborn, W. J. (2012). Crohn's disease. Lancet 380, 1590-1605.
Becker, C., Neurath, M. F., and Wirtz, S. (2015). The Intestinal Microbiota in Inflammatory Bowel Disease. ILAR J 56, 192-204.
Bolger, A. M., Lohse, M., and Usadel, B. (2014). Trimmomatic: a flexible trimmer for Illumina sequence data. Bioinformatics 30, 2114-2120.
Brezinski, E. A., Dhillon, J. S., and Armstrong, A. W. (2015). Economic Burden of Psoriasis in the United States: A Systematic Review. JAMA Dermatol 151, 651-658.
Buchfink, B., Xie, C., and Huson, D. H. (2015). Fast and sensitive protein alignment using DIAMOND. Nat Methods 12, 59-60.
Canani, R. B., Costanzo, M. D., Leone, L., Pedata, M., Meli, R., and Calignano, A. (2011). Potential beneficial effects of butyrate in intestinal and extraintestinal diseases. World J Gastroenterol 17, 1519-1528.
Castro-Rueda, H., and Kavanaugh, A. (2008). Biologic therapy for early rheumatoid arthritis: the latest evidence. Curr Opin Rheumatol 20, 314-319.
Coburn, L. A., Gong, X., Singh, K., Asim, M., Scull, B. P., Allaman, M. M., Williams, C. S., Rosen, M. J., Washington, M. K., Barry, D. P., et al. (2012). L-arginine supplementation improves responses to injury and inflammation in dextran sulfate sodium colitis. PLoS One 7, e33546.
Colombel, J. F., Rutgeerts, P., Reinisch, W., Esser, D., Wang, Y., Lang, Y., Marano, C. W., Strauss, R., Oddens, B. J., Feagan, B. G., et al. (2011). Early mucosal healing with infliximab is associated with improved long-term clinical outcomes in ulcerative colitis. Gastroenterology 141, 1194-1201.
Cross, M., Smith, E., Hoy, D., Carmona, L., Wolfe, F., Vos, T., Williams, B., Gabriel, S., Lassere, M., Johns, N., et al. (2014). The global burden of rheumatoid arthritis: estimates from the global burden of disease 2010 study. Ann Rheum Dis 73, 1316-1322.
D′Haens, G., Baert, F., van Assche, G., Caenepeel, P., Vergauwe, P., Tuynman, H., De Vos, M., van Deventer, S., Stitt, L., Donner, A., et al. (2008). Early combined immunosuppression or conventional management in patients with newly diagnosed Crohn's disease: an open randomised trial. Lancet 371, 660-667.
Eppinga, H., Konstantinov, S. R., Peppelenbosch, M. P., and Thio, H. B. (2014). The microbiome and psoriatic arthritis. Curr Rheumatol Rep 16, 407.
Forbes, J. D., Van Domselaar, G., and Bernstein, C. N. (2016). The Gut Microbiota in Immune-Mediated Inflammatory Diseases. Front Microbiol 7, 1081.
Gevers, D., Kugathasan, S., Denson, L. A., Vazquez-Baeza, Y., Van Treuren, W.,
Ren, B., Schwager, E., Knights, D., Song, S. J., Yassour, M., et al. (2014). The treatment-naive microbiome in new-onset Crohn's disease. Cell Host Microbe 15, 382-392.
Group, N. H. W., Peterson, J., Garges, S., Giovanni, M., McInnes, P., Wang, L., Schloss, J. A., Bonazzi, V., McEwen, J. E., Wetterstrand, K. A., et al. (2009). The NIH Human Microbiome Project. Genome Res 19, 2317-2323.
Haiser, H. J., Gootenberg, D. B., Chatman, K., Sirasani, G., Balskus, E. P., and Turnbaugh, P. J. (2013). Predicting and manipulating cardiac drug inactivation by the human gut bacterium Eggerthella lenta. Science 341, 295-298.
Hamer, H. M., Jonkers, D., Venema, K., Vanhoutvin, S., Troost, F. J., and Brummer, R. J. (2008). Review article: the role of butyrate on colonic function. Aliment Pharmacol Ther 27, 104-119.
Harvey, R. F., and Bradshaw, J. M. (1980). A simple index of Crohn's-disease activity. Lancet 1, 514.
Inan, M. S., Rasoulpour, R. J., Yin, L., Hubbard, A. K., Rosenberg, D. W., and Giardina, C. (2000). The luminal short-chain fatty acid butyrate modulates NF-kappaB activity in a human colonic epithelial cell line. Gastroenterology 118, 724-734.
Integrative, H. M. P. R. N. C. (2014). The Integrative Human Microbiome Project: dynamic analysis of microbiome-host omics profiles during periods of human health and disease. Cell Host Microbe 16, 276-289.
Knights, D., Lassen, K. G., and Xavier, R. J. (2013). Advances in inflammatory bowel disease pathogenesis: linking host genetics and the microbiome. Gut 62, 1505-1510.
Kolios, G., Valatas, V., and Ward, S. G. (2004). Nitric oxide in inflammatory bowel disease: a universal messenger in an unsolved puzzle. Immunology 113, 427-437.
Kostic, A. D., Xavier, R. J., and Gevers, D. (2014). The microbiome in inflammatory bowel disease: current status and the future ahead. Gastroenterology 146, 1489-1499.
Langmead, B., and Salzberg, S. L. (2012). Fast gapped-read alignment with Bowtie 2. Nat Methods 9, 357-359.
Lee, H. S., Han, S. Y., Ryu, K. Y., and Kim, D. H. (2009). The degradation of glycosaminoglycans by intestinal microflora deteriorates colitis in mice. Inflammation 32, 27-36.
Lewis, J. D., Chen, E. Z., Baldassano, R. N., Otley, A. R., Griffiths, A. M., Lee, D.,
Bittinger, K., Bailey, A., Friedman, E. S., Hoffmann, C., et al. (2015). Inflammation, Antibiotics, and Diet as Environmental Stressors of the Gut Microbiome in Pediatric Crohn's Disease. Cell Host Microbe 18, 489-500.
Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079.
Molodecky, N. A., Soon, I. S., Rabi, D. M., Ghali, W. A., Ferris, M., Chernoff, G., Benchimol, E. I., Panaccione, R., Ghosh, S., Barkema, H. W., et al. (2012). Increasing incidence and prevalence of the inflammatory bowel diseases with time, based on systematic review. Gastroenterology 142, 46-54 e42; quiz e30.
Neville, B. A., Sheridan, P. O., Harris, H. M., Coughlan, S., Flint, H. J., Duncan, S. H., Jeffery, I. B., Claesson, M. J., Ross, R. P., Scott, K. P., et al. (2013). Pro-inflammatory flagellin proteins of prevalent motile commensal bacteria are variably abundant in the intestinal microbiome of elderly humans. PLoS One 8, e68919.
Ordas, I., Eckmann, L., Talamini, M., Baumgart, D. C., and Sandborn, W. J. (2012). Ulcerative colitis. Lancet 380, 1606-1619.
Pardee, K., Green, A. A., Ferrante, T., Cameron, D. E., DaleyKeyser, A., Yin, P., and Collins, J. J. (2014). Paper-based synthetic gene networks. Cell 159, 940-954.
Pavlidis, P., Gulati, S., Dubois, P., Chung-Faye, G., Sherwood, R., Bjarnason, I., and Hayee, B. (2016). Early change in faecal calprotectin predicts primary non-response to anti-TNFalpha therapy in Crohn's disease. Scand J Gastroenterol 51, 1447-1452.
Ramasundara, M., Leach, S. T., Lemberg, D. A., and Day, A. S. (2009). Defensins and inflammation: the role of defensins in inflammatory bowel disease. J Gastroenterol Hepatol 24, 202-208.
Ramiro, S., Smolen, J. S., Landewe, R., van der Heijde, D., Dougados, M., Emery, P., de Wit, M., Cutolo, M., Oliver, S., and Gossec, L. (2016). Pharmacological treatment of psoriatic arthritis: a systematic literature review for the 2015 update of the EULAR recommendations for the management of psoriatic arthritis. Ann Rheum Dis 75, 490-498.
Rutgeerts, P., Goboes, K., Peeters, M., Hiele, M., Penninckx, F., Aerts, R., Kerremans, R., and Vantrappen, G. (1991). Effect of faecal stream diversion on recurrence of Crohn's disease in the neoterminal ileum. Lancet 338, 771-774.
Shaw, K. A., Bertha, M., Hofmekler, T., Chopra, P., Vatanen, T., Srivatsa, A., Prince, J., Kumar, A., Sauer, C., Zwick, M. E., et al. (2016). Dysbiosis, inflammation, and response to treatment: a longitudinal study of pediatric subjects with newly diagnosed inflammatory bowel disease. Genome Med 8, 75.
Shelton, E., Allegretti, J. R., Stevens, B., Lucci, M., Khalili, H., Nguyen, D. D., Sauk, J., Giallourakis, C., Garber, J., Hamilton, M. J., et al. (2015). Efficacy of Vedolizumab as Induction Therapy in Refractory IBD Patients: A Multicenter Cohort. Inflamm Bowel Dis 21, 2879-2885.
Siegel, C. A., and Melmed, G. Y. (2009). Predicting response to Anti-TNF Agents for the treatment of crohn's disease. Therap Adv Gastroenterol 2, 245-251.
Singh, J. A., Saag, K. G., Bridges, S. L., Jr., Akl, E. A., Bannuru, R. R., Sullivan, M. C., Vaysbrot, E., McNaughton, C., Osani, M., Shmerling, R. H., et al. (2016). 2015 American College of Rheumatology Guideline for the Treatment of Rheumatoid Arthritis. Arthritis Rheumatol 68, 1-26.
Singh, S., Graff, L. A., and Bernstein, C. N. (2009). Do NSAIDs, antibiotics, infections, or stress trigger flares in IBD? Am J Gastroenterol 104, 1298-1313; quiz 1314.
Truong, D. T., Franzosa, E. A., Tickle, T. L., Scholz, M., Weingart, G., Pasolli, E., Tett, A., Huttenhower, C., and Segata, N. (2015). MetaPhlAn2 for enhanced metagenomic taxonomic profiling. Nat Methods 12, 902-903.
Ungaro, R., Bernstein, C. N., Gearry, R., Hviid, A., Kolho, K. L., Kronman, M P., Shaw, S., Van Kruiningen, H., Colombel, J. F., and Atreja, A. (2014). Antibiotics associated with increased risk of new-onset Crohn's disease but not ulcerative colitis: a meta-analysis. Am J Gastroenterol 109, 1728-1738.
Upchurch, K. S., and Kay, J. (2012). Evolution of treatment for rheumatoid arthritis. Rheumatology (Oxford) 51 Suppl 6, vi28-36.
Walmsley, R. S., Ayres, R. C., Pounder, R. E., and Allan, R. N. (1998). A simple clinical colitis activity index. Gut 43, 29-32.
Wu, H. J., Ivanov, I, Darce, J., Hattori, K., Shima, T., Umesaki, Y., Littman, D. R., Benoist, C., and Mathis, D. (2010). Gut-residing segmented filamentous bacteria drive autoimmune arthritis via T helper 17 cells. Immunity 32, 815-827.
Zimmerman, M. A., Singh, N., Martin, P. M., Thangaraju, M., Ganapathy, V., Waller, J. L., Shi, H., Robertson, K. D., Munn, D. H., and Liu, K. (2012). Butyrate suppresses colonic inflammation through HDAC1-dependent Fas upregulation and Fas-mediated apoptosis of T cells. Am J Physiol Gastrointest Liver Physiol 302, G1405-1415.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method of treating a selected subject with an inflammatory bowel disease (IBD), comprising administering to the subject an anti-integrin therapy, wherein the subject is selected as having increased levels of Roseburia inulinivorans and/or a Burkholdenales species as compared to a control subject.

2. The method of claim 1, wherein IBD is Crohn's disease (CD), ulcerative colitis (UC)), rheumatoid arthritis (RA), or psoriasis (PsA).

3. The method of claim 2 further comprising measuring levels of one or more metabolic pathways selected from the group consisting of super-pathway of arginine and polyamine biosynthesis; super-pathway of branched amino acid biosynthesis; Calvin-Benson-Bassham cycle; L-citrulline biosynthesis; dTDP-L-rhamnose biosynthesis I; super-pathway of N-acetyleglucosamine, N-acetylmannosamin and N-acetylneuraminate degradation; super-pathway of β-D-glucuronide and D-glucuronate degradation; super-pathway of hexitol degradation; L-isoleucine biosynthesis I; super-pathway of polyamine biosynthesis I; L-histidine degradation III; GDP-mannose biosynthesis; acetyl-CoA fermentation to butanoate II; colonic acid building blocks biosynthesis; lipid IVA biosysnthesis; N10-formyl-tetrahydrofolate biosysnthesis; pentose phosphate pathway, and pyruvate fermentation to acetate and lactate II; as compared to a subject with higher baseline levels of N10-formyl-tetrahydrofolate biosynthesis, pentose phosphate pathway; and/or pyruvate fermentation to acetate and lactate II has an increased likelihood of responding to treatment for UC.

4. (canceled)

5. The method of claim, wherein the anti-integrin therapy is vedolizumab.
